(19) United States
(12) Patent Application Publication
(10) Pub. No.: US 2025/0114599 A1
(43) Pub. Date: Apr. 10, 2025

(54) FULLY COLLAPSABLE TDCS/TACS APPLICATION DEVICE WITH AUDIO

(71) Applicant: VYSRE, Inc., Gilbert, AZ (US)

(72) Inventors: MATT STEPHEN HAMEL, Gilbert, AZ (US); CHUNG QUOC TRINH, Gilbert, AZ (US)

(21) Appl. No.: 18/924,667

(22) Filed: Oct. 23, 2024

Related U.S. Application Data
(63) Continuation of application No. PCT/US23/20478, filed on Apr. 28, 2023.
(60) Provisional application No. 63/336,942, filed on Apr. 29, 2022.

(51) Int. Cl. A61N 1/36; A61N 1/04

(52) U.S. Cl. CPC A61N 1/36031; A61N 1/0472

(57) ABSTRACT

The presented invention relates to the application of low current temporal Direct Current Stimulation and a simulated alternating current to achieve Alternating Current Stimulation and Random Noise Stimulation to different regions of the brain to enhance brain function and activity. More specifically, the invention is designed to target and stimulate several specific locations of the brain, separately or concurrently, to produce a short-term or long-term positive effect on a user's brain. This invention is designed to deliver short and long duration stimulations at varying frequencies through specialized nodules embedded in a headset enclosure to produce effect. The enclosure of the headset will also have an integrated microphone audio component and virtual reality (VR) component to enable audio and visual stimulation in conjunction with the electrical stimulation.

### CROSS-REFERENCE

This application claims the benefit of PCT Application No. PCT/US23/20478, filed Apr. 28, 2023, which claims the benefit of U.S. Provisional Application No. 63/336,942, filed Apr. 29, 2022, which is hereby incorporated by reference in its entirety herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

High current or power devices, such as Transcranial Magnetic Stimulation (TMS) producing neuromodulation and non-invasive brain stimulation devices, have been recognized and cleared by the FDA for use in treating depression and other mental health disorders. These devices have been shown to produce brain activation at the cellular and synaptic level. However, there is a growing need to provide low-powered devices which may help with overall brain wellness, sharpness, and acuity. For example, the application of low current temporal stimulation like transcranial Direct Current Stimulation (tDCS) is a fast-growing technology which has been shown to notably improve various brain functions, such as cognition and mood.

The low-powered device market currently is split into two types of non-invasive transcranial stimulation devices. There are tDCS systems which are either handheld or head-mounted, and transcranial Alternating Current Stimulation (tACS) systems that are predominantly handheld. In both cases, these devices are not designed for extended wear. Further, these low-power devices utilize wet electrodes which require the use of a saline salt solutions to provide an electrical connection between the system and the user.

Other devices make use of dry electrodes, but the electrodes only implement a single direction current flow without the ability to change in direction of the current flow between electrodes.

Therefore, there are needs for a low-powered system that can achieve several different types of low current temporal stimulation such as tDCS, tACS, tRNS, and even more customized waveforms, to be delivered to different parts of the brain, at different sequences of pattern, at the same time, different time, in synchronized or non-synchronized patterns.

### SUMMARY

In an aspect, the present disclosure provides a system for applying transcranial stimulation to a user. In some embodiments, the system may comprise a first set of one or more pairs of electrodes coupled to a first adjustable band. The first adjustable band may be configured to allow the first set of one or more pairs of electrodes to be positioned onto a first region of a user's head. In some embodiments, the system may comprise a second set of one or more pairs of electrodes coupled to a second adjustable band. The second adjustable band may be configured to allow the second set of one or more pairs of electrodes to be positioned onto a second region of the user's head. In some embodiments, the first and second regions of the user's head may be selected on a desired transcranial stimulation. In some embodiments, the system may comprise a circuitry electronically connected to the first and second set of one or more pairs of electrodes. The circuitry may be configured to generate a waveform for the desired transcranial stimulation of the user. The circuitry may be configured to transmit the waveform to the first and second set of one or more pairs of electrodes to provide the desired transcranial stimulation of the first and second regions of the user's head.

In some embodiments, the first and second regions may be selected from a group comprising the frontal lobe (prefrontal cortex), the cerebral cortex, the motor cortex, the parietal lobe, and the occipital lobe.

In some embodiments, the circuitry may be configured to select individual electrodes of the first set of one or more pairs of electrodes and the second set of one or more pair of electrodes as inactive, active and anodic, or active and cathodic.

In some embodiments, each electrode of the first set of one or more pairs of electrodes may be either an anode or a cathode. In other embodiments, all electrodes of the first set of one or more pairs of electrodes may be either an anode or a cathode.

In some embodiments, each electrode of the first set of one or more pairs of electrodes may have a dedicated ground.

In some embodiments, each electrode of the first set of one or more pairs of electrodes may have one or more node clusters comprising a plurality of contacts. Each of the plurality of contacts independently may be either active or inactive.

In some embodiments, each active contact of the plurality of contacts may be configured to provide an electrical connection between the circuitry and the first region of the user's head.

In some embodiments, the number of active contacts of the plurality of contacts may be proportional to a desired charge density.

In some embodiments, each electrode of the first set of one or more pairs of electrodes may be adjustable to minimize a distance between the plurality of contacts and the first region of the user's head.

In some embodiments, each electrode of the first set of one or more pairs of electrodes may be configured to measure brainwave activity near the first region of the user's head.

In some embodiments, the system may use an internal reference to verify proper positioning of the first set of one or more pairs of electrodes at the first region of the user's head.

In some embodiments, each electrode of the second set of one or more pairs of electrodes may be either an anode or a cathode. In other embodiments, all electrodes of the second set of one or more pairs of electrodes may be either an anode or a cathode.

In some embodiments, each electrode of the second set of one or more pairs of electrodes may have a dedicated ground.

In some embodiments, each electrode of the second set of one or more pairs of electrodes may have one or more node clusters comprising a plurality of contacts. Each of the plurality of contacts independently may be either active or inactive.

In some embodiments, each active contact of the plurality of contacts may be configured to provide an electrical connection between the circuitry and the second region of the user's head.

In some embodiments, the number of active contacts of the plurality of contacts may be proportional to a desired charge density.

Each electrode of the second set of one or more pairs of electrodes may be adjustable to minimize a distance between the plurality of contacts and the second region of the user's head.

In some embodiments, each electrode of the second set of one or more pairs of electrodes may be configured to measure brainwave activity near the second region of the user's head.

In some embodiments, the system may use an internal reference to verify proper positioning of the second set of one or more pairs of electrodes at the second region of the user's head.

In some embodiments, the system may be configured to apply either transcranial direct current stimulation (tDCS), transcranial alternating current stimulation (tACS), transcranial random noise stimulation (tRNS), transcranial variable frequency stimulation (tVFS), and transcranial Complex Waveform Stimulation (tCWS). Each electrode of the first set of one or more pairs of electrodes and each electrode of the second set of one or more pairs of electrodes may be independently configured to apply either transcranial direct current stimulation (tDCS), transcranial alternating current stimulation (tACS), transcranial random noise stimulation (tRNS), transcranial variable frequency stimulation (tVFS), and transcranial Complex Waveform Stimulation (tCWS).

In some embodiments, the system may be configured to provide the desired transcranial stimulation bilaterally. Providing the desired transcranial stimulation bilaterally may comprise applying a different transcranial stimulation to the left and right hemispheres of the user's head. Providing the desired transcranial stimulation bilaterally may comprise applying, independently to each of the left and right hemispheres, either transcranial direct current stimulation (tDCS), transcranial alternating current stimulation (tACS), transcranial random noise stimulation (tRNS), transcranial variable frequency stimulation (tVFS), and transcranial Complex Waveform Stimulation (tCWS).

In some embodiments, the system may be configured to provide the desired transcranial stimulation cross laterally.

In some embodiments, the system may be configured to reverse the direction of the desired transcranial stimulation.

In some embodiments, the circuitry may be configured to modulate one or more of a frequency, amplitude, and centerline offset of the waveform for the desired transcranial stimulation of the user.

In some embodiments, the circuitry may be configured to alternate between transcranial direct current stimulation (tDCS), transcranial alternating current stimulation (tACS), transcranial random noise stimulation (RNS), transcranial variable frequency stimulation (tVFS), and transcranial Complex Waveform Stimulation (tCWS) to provide the desired transcranial stimulation.

In some embodiments, the desired transcranial stimulation may be either constant or pulsed.

In some embodiments, the circuitry may be configured to interface with a computer.

In some embodiments, the computer may be configured to allow the user input qualitative data.

In some embodiments, the computer may be configured to allow the user to select the desired transcranial stimulation.

In some embodiments, the computer may be configured to provide instructions to the user for positioning the first and second set of one or more pairs of electrodes on the first and second regions of the user's head.

In some embodiments, the position of the first and second adjustable bands relative to one another may be adjustable.

In some embodiments, the system may further comprises an audio component.

In some embodiments, the system may further comprise a visual component.

In another aspect, the present disclosure provides a method for providing transcranial stimulation to a user. In some embodiments, the method may comprise the step of providing, to a user, instructions for positioning an adjustable transcranial stimulation device on the user's head to contact a first and a second region of the user's head. The first and second region of the user's head may be selected on a desired transcranial stimulation. In some embodiments, the method may comprise the step of applying the desired transcranial stimulation to apply a neuromodulation to the user. In some embodiments, the method may comprise the step of receiving one or more of qualitative or quantitative feedback from the user in response to the neuromodulation. In some embodiments, the method may comprise the step of adjusting the desired transcranial stimulation in response to the one or more of a qualitative or quantitative feedback from the user. In some embodiments, the method may comprise the step of applying the adjusted desired transcranial stimulation with the adjustable transcranial stimulation device.

In some embodiments, the first and second regions may be selected from a group comprising the frontal lobe (prefrontal cortex), the cerebral cortex, the motor cortex, the parietal lobe, and the occipital lobe.

In some embodiments, applying the desired transcranial stimulation may comprise selecting each electrode of a first set of one or more pairs of electrodes and a second set of one or more pairs of electrodes of the adjustable transcranial device as inactive, ground, active and anodic, or active and cathodic.

In some embodiments, the method further comprises determining the position of each electrode of a first set of one or more pairs of electrodes of the adjustable transcranial stimulation device using an internal reference.

In some embodiments, the method further comprises providing instructions to the user for adjusting the position of at least one electrode of the first set of one or more pairs of electrodes in response to the determined position.

In some embodiments, the method further comprises adjusting the position of at least one electrode of the first set of one or more pairs of electrodes.

In some embodiments, adjusting the position of at least one electrode of the first set of one or more pairs of electrodes may comprise adjusting a position, on the user's head, of an adjustable band coupled to the at least one electrode of the first set of one or more pairs of electrodes.

In some embodiments, the method further comprises determining the position of each electrode of a second set of one or more pairs of electrodes of the adjustable transcranial stimulation device using an internal reference.

In some embodiments, the method further comprises providing instructions to the user for adjusting the position of at least one electrode of the second set of one or more pairs of electrodes in response to the determined position.

In some embodiments, the method further comprises adjusting the position of at least one electrode of the second set of one or more pairs of electrodes.

In some embodiments, receiving qualitative feedback from a user may comprise receiving user responses to a survey provided to the user through an app on a computing device.

In some embodiments, the survey may request the user to provide feedback on one or more of the user's mood, activity, quality of life, experience, attention, concentration, coordination, information processing, activity speed, or other subjective measurable data.

In some embodiments, receiving qualitative feedback may occur prior to applying the desired transcranial stimulation.

In some embodiments, receiving qualitative feedback may occur after applying the desired transcranial stimulation.

In some embodiments, receiving quantitative feedback from a user may comprise measuring one or more physiological vital metrics of the user. The one or more physiological vital metrics may comprise heart rate.

In some embodiments, receiving quantitative feedback from a user may comprise measuring electrical activity from the brain of the user.

In some embodiments, receiving quantitative feedback may occur prior to applying the desired transcranial stimulation.

In some embodiments, receiving quantitative feedback may occur after applying the desired transcranial stimulation.

In some embodiments, adjusting the desired transcranial stimulation may comprise processing the one or more of a qualitative or quantitative feedback from the user using a machine learning algorithm.

In some embodiments, each of the one or more of a qualitative or quantitative feedback may be weighted by the machine learning algorithm.

In some embodiments, the machine learning algorithm may use real-time and historical data.

In some embodiments, adjusting the desired transcranial stimulation may comprise adjusting at least one electrode of a first set of one or more pairs of electrodes of the adjustable transcranial stimulation device.

In some embodiments, adjusting the desired transcranial stimulation may comprise adjusting at least one electrode of a second set of one or more pairs of electrodes of the adjustable transcranial stimulation device.

In some embodiments, adjusting the desired transcranial stimulation may comprise adjusting a waveform generated by a circuitry of the adjustable transcranial stimulation device.

In some embodiments, adjusting a waveform may comprise adjusting one or more of a frequency, amplitude, and centerline offset of the waveform.

In some embodiments, adjusting the desired transcranial stimulation may comprise alternating between transcranial direct current stimulation (tDCS), transcranial alternating current stimulation (tACS), transcranial random noise stimulation (tRNS), transcranial variable frequency stimulation (tVFS), and transcranial Complex Waveform Stimulation (tCWS).

In some embodiments, adjusting the desired transcranial stimulation may comprise reversing a direction of the desired transcranial stimulation.

In some embodiments, the desired transcranial stimulation may be applied bilaterally.

In some embodiments, bilateral stimulation may comprise applying a different transcranial stimulation to the left and right hemispheres of the user's head.

In some embodiments, the bilateral stimulation may comprise applying independently to each of the left and right hemispheres either transcranial direct current stimulation (tDCS), transcranial alternating current stimulation (tACS), transcranial random noise stimulation (tRNS), transcranial variable frequency stimulation (tVFS), and transcranial Complex Waveform Stimulation (tCWS).

In some embodiments, the desired transcranial stimulation may be applied cross laterally.

In some embodiments, applying a neuromodulation to a user may comprise enhancing the user's coordination or concentration.

In some embodiments, applying a neuromodulation to a user may comprise enhancing either a brain's wellness or a brain's health.

In some embodiments, applying a neuromodulation to a user may comprise enhancing a brain's ability to process information.

In some embodiments, applying a neuromodulation to a user may comprise enhancing the user's task performance or acuity.

In some embodiments, the instructions may be provided to the user using a computer connected to the adjustable transcranial stimulation device.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 are drawings illustrating bilateral and cross lateral transcranial stimulation, according to some embodiments.

FIG. 2 is a first perspective view of an adjustable transcranial stimulation headset in a collapsed state, according to some embodiments.

FIG. 3 is a second perspective view of an adjustable transcranial stimulation headset in a deployed state, according to some embodiments.

FIGS. 4a and 4b are close-up views of a rotation piece and earpad, respectively, of an adjustable transcranial stimulation headset, according to some embodiments.

FIG. 5 is an exploded view of an adjustable transcranial stimulation headset, according to some embodiments.

FIGS. 6a and 6b are close-up views of an electrode assembly with node clusters retracted and deployed, respectively, of an adjustable transcranial stimulation headset, according to some embodiments.

FIG. 7 is an exploded view of an electrode assembly of an adjustable transcranial stimulation headset, according to some embodiments.

FIGS. 8a and 8b are graphs illustrating the sine wave for the resistors of the logic gate, and further exemplify how the sine wave of the resistors of the logic gate can be modulated to change the frequency and the amplitude, according to some embodiments.

FIGS. 9a and 9b are graphs illustrating how the random noise intensity and amplitude can be varied according to the functionality of the microcontroller, according to some embodiments.

FIG. 10 is a graph showing a complex wave form generated by the microcontroller, according to some embodiments.

FIG. 11 is a diagram showing a circuitry of an adjustable transcranial stimulation headset, according to some embodiments.

FIGS. 12a and 12b are a schematic and a diagram showing non-limiting examples of the circuitry of an adjustable transcranial stimulation headset, according to some embodiments.

FIG. 13 is a drawing depicting the regions of the head of a user which may be stimulated using the adjustable transcranial stimulation headset, according to some embodiments.

FIGS. 14a and 14b are drawings illustrating a side view of the adjustable transcranial stimulation headset positioned on the head of a user in a collapsed state and deployed state, respectively, according to some embodiments.

FIG. 15 is a drawing illustrating a top-down view of the adjustable transcranial stimulation headset positioned on the head of a user in a deployed state, according to some embodiments.

FIG. 16 is a flow chart illustrating an exemplary method for transcranial stimulation using the adjustable transcranial stimulation headset, according to some embodiments.

FIG. 17 is a drawing illustrating data flow to and from the adjustable transcranial stimulation headset via a computer, according to some embodiments.

FIG. 18 shows a non-limiting example of a computing device; in this case, a device with one or more processors, memory, storage, and a network interface, according to some embodiments.

FIG. 19 shows a non-limiting example of a web/mobile application provision system; in this case, a system providing browser-based and/or native mobile user interfaces, according to some embodiments.

FIG. 20 shows a non-limiting example of a cloud-based web/mobile application provision system; in this case, a system comprising an elastically load balanced, auto-scaling web server and application server resources as well synchronously replicated databases, according to some embodiments.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Low current temporal stimulation has shown to have a mild, yet modulatory, effect on brain function and activity. Provided herein are low-powered devices for transcranial stimulation and methods for use. These devices may provide a safe and gentle stimulation that can modulate and build upon the brain's function. Modulation may be accomplished through use of different types of stimulation such as transcranial Direct Current Stimulation (tDCS), transcranial Alternating Current Stimulation (tACS), transcranial Random Noise Stimulation (tRNS), transcranial Variable Frequency Stimulation (tVFS), and transcranial Complex Waveform Stimulation (tCWS). Further, modulation may be adjusted through changes in placement of electrodes, energy levels, frequency, and sequence pattens. The modulatory effect of the devices described herein is targeted to select regions of the brain, such as the dorsolateral prefrontal cortex, medial prefrontal cortex, lateral prefrontal cortex, premotor cortex, supplemental motor cortex, occipital cortex, limbic system, hippocampus, amygdala, posterior parietal cortex, parietal cortex, temporal cortex, superior temporal gyrus, visual spatial pathway, and other internetwork pathways.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

### Adjustable Transcranial Stimulation System

Provided herein are systems for applying transcranial stimulation to a user.

The transcranial stimulation system is configured to apply either transcranial Direct Current Stimulation (tDCS), transcranial Alternating Current Stimulation (tACS), transcranial Random Noise Stimulation (tRNS), transcranial Variable Frequency Stimulation (tVFS), transcranial Complex Waveform Stimulation (tCWS), or combinations thereof.

The transcranial stimulation system may stimulate discrete regions of the brain such as the dorsolateral prefrontal cortex, medial prefrontal cortex, lateral prefrontal cortex, premotor cortex, supplemental motor cortex, occipital cortex, limbic system, hippocampus, amygdala, posterior parietal cortex, parietal cortex, temporal cortex, superior temporal gyrus, visual spatial pathway, and other internetwork pathways.

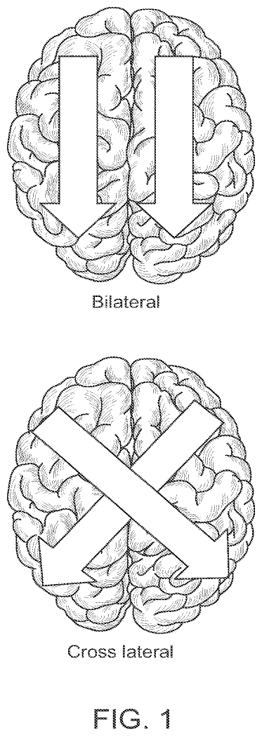

In addition, the transcranial stimulation system may provide transcranial stimulation bilaterally (as shown in FIG. 1), such that each of the left and right hemispheres of the brain may independently receive either transcranial Direct Current Stimulation (tDCS), transcranial Alternating Current Stimulation (tACS), transcranial Random Noise Stimulation (tRNS), transcranial Variable Frequency Stimulation (tVFS), transcranial Complex Waveform Stimulation (tCWS), or combinations thereof.

Further, the transcranial stimulation system may provide transcranial stimulation cross laterally (also shown in FIG. 1), such that a stimulation originating in the left hemisphere of the brain crosses a stimulation originating in the right hemisphere of the brain. Cross lateral stimulation may comprise either transcranial Direct Current Stimulation (tDCS), transcranial Alternating Current Stimulation (tACS), transcranial Random Noise Stimulation (tRNS), transcranial Variable Frequency Stimulation (tVFS), transcranial Complex Waveform Stimulation (tCWS), or combinations thereof.

### Adjustable Stimulation Headset

In some instances, the transcranial stimulation system comprises an adjustable non-invasive head-mounted low-current temporal stimulation device (i.e., an adjustable stimulation headset). The adjustability of the transcranial stimulation system described here can be intended to facilitate an electrical connection between the headset and the user. An improved electrical connection between the headset and the user may improve the efficiency of the intended stimulation. The adjustability of the headset described herein can allow for the simultaneous stimulation of one or more regions of the user's head. For example, the headset may be configured to stimulate a first and a second region of the user's head. The first and second regions of the user's head may correspond to a desired transcranial stimulation. The first and second regions may be selected from a group comprising the frontal lobe (prefrontal cortex), the cerebral cortex, the motor cortex, the parietal lobe, and the occipital lobe.

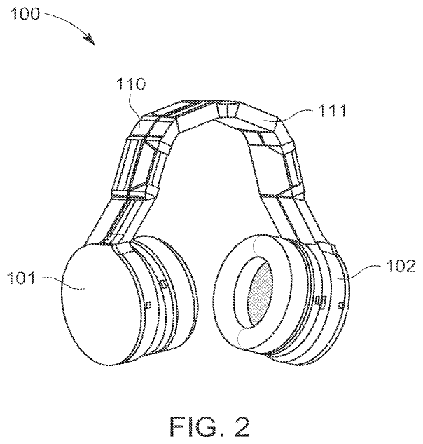

As shown in FIGS. 2, 3, and 4, embodiments of the adjustable stimulation headset 100 may comprise a first set of one or more pairs of electrodes 120 coupled to a first adjustable band 110. The first adjustable band 110 can be configured to allow the first set of one or more pairs of electrodes 120 to be positioned onto a first region of the user's head. The first set of one or more pairs of electrodes 120 may be located either symmetrically or asymmetrically on the first adjustable band 110. The transcranial stimulation system 100 further comprises a second set of one or more pairs of electrodes 121 coupled to a second adjustable band 111. The second adjustable band 111 can be configured to allow the second set of one or more pairs of electrodes 121 to be positioned onto a second region of the user's head. The second set of one or more pairs of electrodes 121 may be located either symmetrically or asymmetrically on the second adjustable band 111.

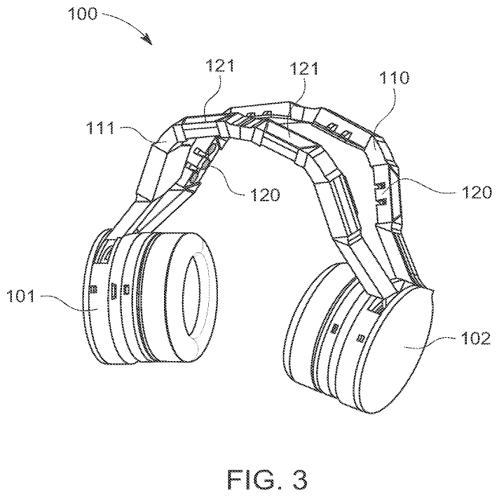

As shown in FIGS. 2 and 3, the adjustable stimulation headset 100 may be configured to transition between a collapsed state (as shown in FIG. 2) and a deployed state (as shown in FIG. 3). In the collapsed state, the first adjustable band 110 and the second adjustable band may be positioned adjacent to one another (i.e., flush with one another), such that a first set of one or more pairs of electrode covers on the first adjustable band 110 is situated over the second set of one or more pairs of electrodes 121. Further, in the collapsed state, a second set of one or more pairs of electrode covers on the second adjustable band 111 may be situated over the first set of one or more pairs of electrodes 120. In the deployed state, the first set of one or more pairs of electrodes 120 and second set of one or more pairs of electrodes 121 may be exposed (i.e., uncovered), allowing the electrodes to interface (i.e., electronically couple) with the first and second regions of the user's head. Deployment of the adjustable stimulation headset 100 can cause node clusters of the first set of one or more pairs of electrodes 120 and second set of one or more pairs of electrodes 121 to extend through openings in the node housings.

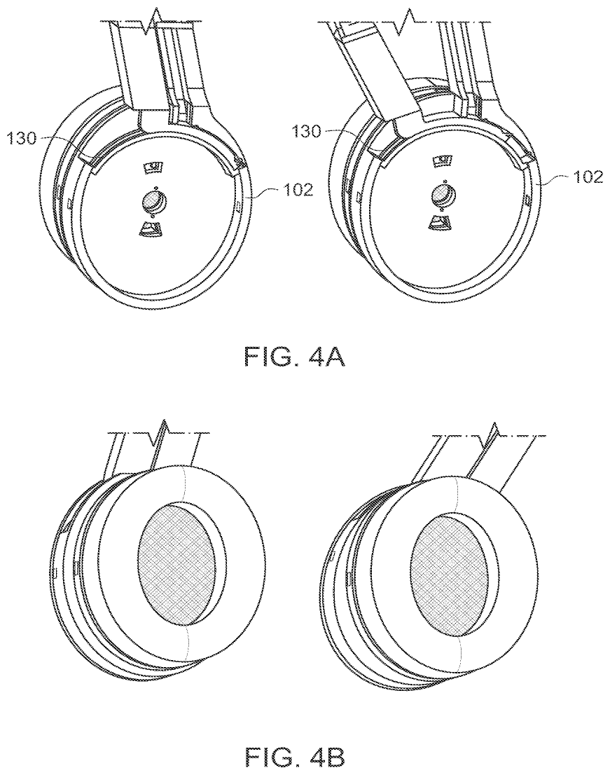

The adjustable stimulation headset 100 may comprise a mechanism for transitioning between the collapsed state and the deployed state. For example, as shown in FIGS. 4a and 4b, the system may include a left headphone assembly 101 and right headphone assembly 102 each independently having a track 130 (or rail) configured to mechanically couple with the second adjustable band 111 in a manner that allows the second adjustable band 111 to slide (i.e., extend or protract) along the track 130 causing the second adjustable band 111 to separate from the first adjustable band 110. The track 130 may act like a pivot point cause separation of the first adjustable band 110 and second adjustable band 111 to occur in a rotational or circular manner. The mechanism for transitioning the adjustable stimulation headset 100 between a collapsed state and a deployed state may be configured to allow a user to effect a transition while wearing (i.e., during a stimulation cycle) the adjustable stimulation headset 100. The track 130 may include a friction source which prevents the inadvertent movement of the first adjustable band 110 and second adjustable band 111. While it is described that the adjustable stimulation headset 100 can be transitioned between collapsed and deployed states, the adjustable stimulation headset 100 can be held in a state between being fully collapsed and fully deployed so that the first adjustable band 110 and the second adjustable band 111 can be positioned to various regions of the user's head.

Referring back to the track 130, the friction source may be a plurality of indentations (not shown) disposed along the track 130. The plurality of indentations may serve to secure the first adjustable band 110 and second adjustable band 111 in a fixed position during use (i.e., during a stimulation cycle). The plurality of indentations may further serve as a means for identifying a degree of separation between the first adjustable band 110 and the second adjustable band 111, and by extension a degree of separation between the first set of one or more pairs of electrodes 120 and the second set of one or more pairs of electrodes 121. For example, the plurality of indentations can denote an angle in degrees between the first adjustable band 110 and the second adjustable band 111. The angle denoted by the plurality of indentations may be from about 0 degrees) (°) to about 90°. The angle denoted by the plurality of indentations may be about 0°, 5°, 10°, 20°, 30°, 40°, 50°, 60°, 70°, 80°, 90°, or any values therebetween. The angle denoted by the plurality of indentations may correspond to a position of the first adjustable band 110 and the second adjustable band 111 with respect to the first and second regions of the user's head. The plurality of indentations may be positioned at either regular or irregular intervals along the track 130.

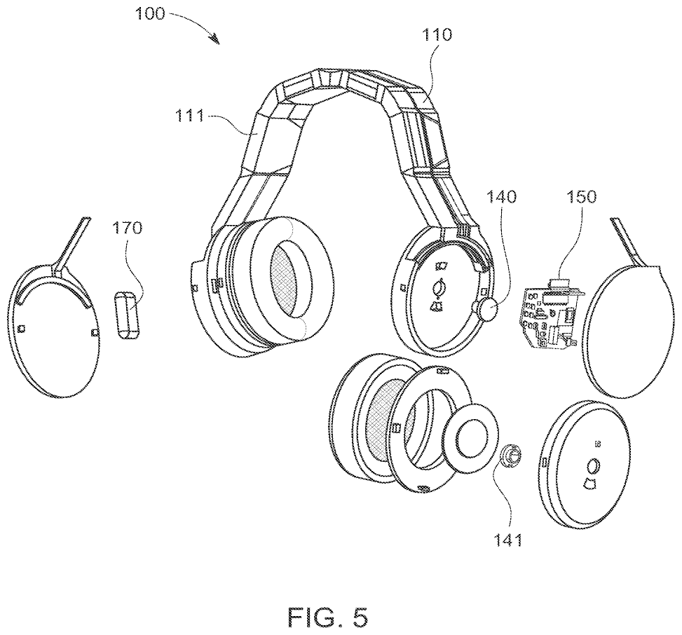

As shown in FIG. 5, the adjustable stimulation headset 100 has a mechanism (e.g., a ball joint, radial bearing, or radial coupler) for incrementally adjusting a position of the first adjustable band 110 and second adjustable band 111. For example, the left headphone assembly 101 and right headphone assembly 102 may comprise a ball joint (male ball joint connector 140 and female ball joint connector 141) configured to allow the first adjustable band 110 and second adjustable band 111 to be rotated, relative to the left headphone assembly 101 and right headphone assembly 102, such that a user may incrementally adjust the position of the first adjustable band 110 on the first region of the user's head and/or the second adjustable band 111 on the second region of the user's head. The ball joint may be configured to allow the first adjustable band 110 and second adjustable band 111 to be rotated either anteriorly or posteriorly.

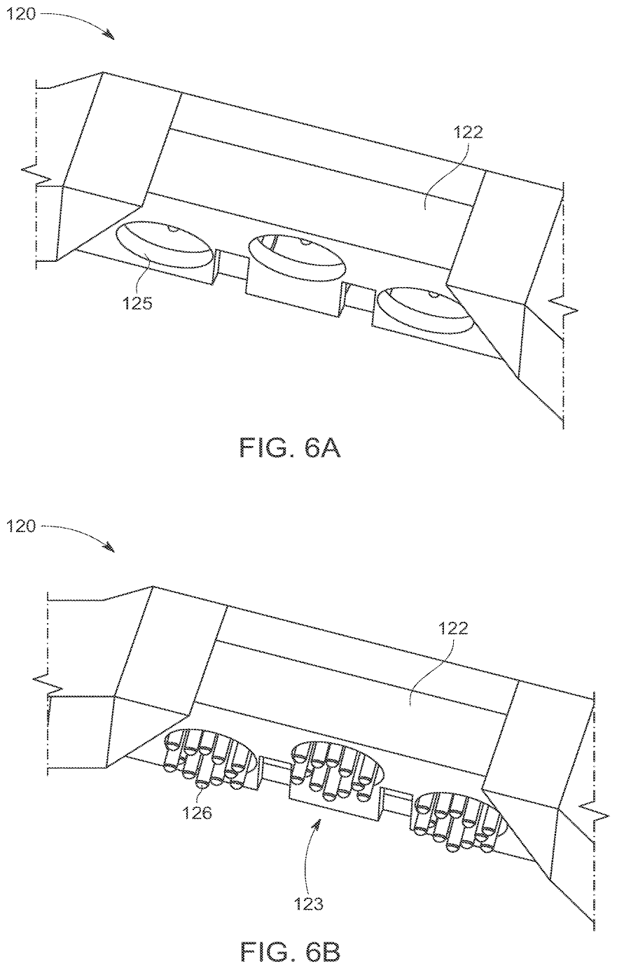

As shown in FIGS. 6a and 6b, each electrode of the first set of one or more pairs of electrodes 120 and second set of one or more pairs of electrodes 121 comprises a node housing 122 having one or more node clusters 123 disposed therein. The node housing 122 may comprise 1, 2, 3, 4, 5, 6, 7, 8, or 9 node clusters 123. The node clusters 123 may be arranged in any manner within the node housing 122. For example, the node clusters 123 may be arranged linearly or offset.

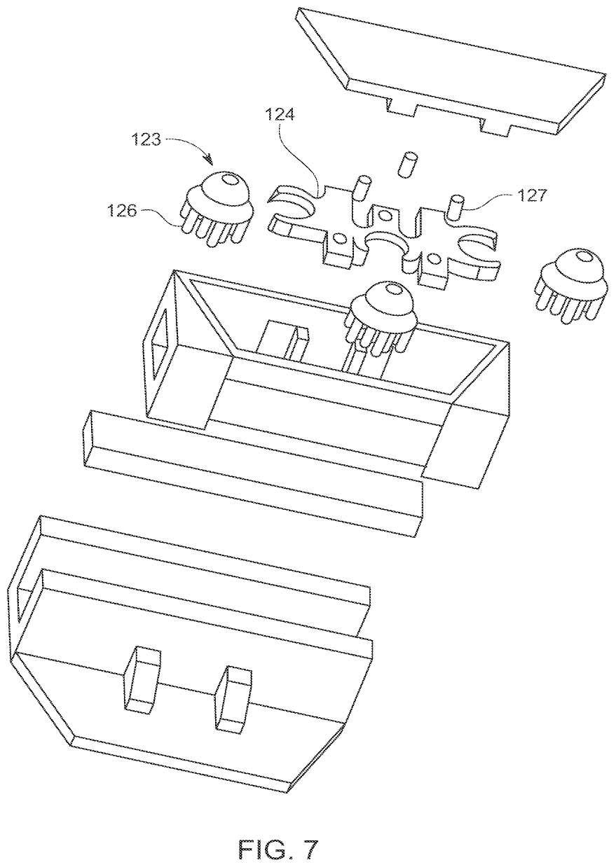

The node housing 122 may further comprise a mechanism for extending and retracting the one or more node clusters 123 through one or more openings 125 in the node housing 122. For example, as shown in FIG. 7, the one or more node clusters 123 may be attached to a node holder 124 which is configured to slide along rails (or tracks) within the node housing 122. One or more springs 127 may be configured to apply a downward pressure onto the node holder 124 causing the one or more node clusters 123 to extend from the one or more openings 125 in the node housing 122. The one or more node clusters 123 may be depressed (i.e., the one or more springs 127 may be depressed) into the node housing 122 via a pressure applied by the first set of one or more pairs of electrode covers and second set of one or more pairs of electrode covers. The node covers may be intended to secure the node clusters 123 to prevent unwanted contact when the transcranial stimulation is not being applied. The retractability (i.e., depression) of the node clusters 123 can improve comfort of the adjustable stimulation headset 100 and allows the headset to be worn for extended periods, including when not being used for transcranial stimulation.

Each node cluster 123 may comprise a plurality of contacts 126. In some embodiments, the plurality of contacts 126 comprises 2, 3, 4, 5, 6, 7, 8, 9, 10, or more contacts. Each of the plurality of contacts 126 independently may be either active or inactive. Each active contact of the plurality of contacts 126 may provide an electrical connection between the adjustable stimulation headset 100 and the user. All contacts of the plurality of contacts 126 in each node cluster 123 may be active, some contacts of the plurality of contacts 126 in each node cluster 123 may be active, or all contacts of the plurality of contacts 126 in each node cluster 123 may be inactive. For each electrode, the active contacts in all node clusters 123 form a contact area to facilitate an electrical connection between the adjustable stimulation headset 100 and the user. Further, by controlling the number of active contacts in all node clusters 123 of an electrode, the size of the contact area may be controlled. The greater the number of active contacts in all node clusters 123 of an electrode, the greater the size of the contact area. The lower the number of active contacts in all node clusters 123 of an electrode, the lower the size of the contact area. The size of the contact area may correspond to the size of a stimulation region but does not necessarily have to correspond to the size of a stimulation region. For example, the stimulation region may be either smaller or larger than the contact area.

Generally, the contact area defined by the active contacts all node clusters 123 of an electrode may be from about 0.5 cm² to about 40 cm² or larger. The contact area may be 0.5 cm², 1 cm², 2 cm², 4 cm², 6 cm², 8 cm², 10 cm², 15 cm², 20 cm², 30 cm², 40 cm², or larger.

The adjustable stimulation headset 100 may be configured to select the number of active contacts in all node clusters 123 of an electrode based on a desired transcranial stimulation, thus the total contact area is a function of the desired transcranial stimulation. The number of active contacts of in all node clusters 123 of an electrode may be proportional to a desired charge density. The desired charge density may be from about 50 coulombs per square meter (C/m²) to about 2000 C/m². In some embodiments, the desired charge density is about 50 C/m², 100 C/m², 150 C/m², 200 C/m², 250 C/m², 300 C/m², 400 C/m², 500 C/m², 750 C/m², 1000 C/m², 1500 C/m², 2000 C/m², and any values therebetween. The desired charge density may take into account a need to limit skin irritation and brain lesions due to heat, and the charge density will vary based on the duration of stimulation, type of stimulation, and stimulation intensity or power. The adjustable stimulation headset 100 may be configured to determine the resistance across all the node clusters 123 of an electrode and may reduce the number of active contacts to ensure the max charge density for a desired transcranial stimulation is not exceeded.

Each node cluster 123 may be made from a conductive material. Each node cluster 123 may comprise a metal (e.g., gold, steel, platinum), a metal alloy (e.g., gold alloy, platinum alloy), a semiconductor (e.g., silicon, doped silicon, a carbon-based semiconductor), a conductive polymer (e.g., polyacetylene, polyphenylene vinylene, polythiophene, polyaniline, polyphenylene sulfide, polypyrrole), any other suitable conductive material, or combinations of any of the foregoing. The conductive material may be configured to provide an electrical connection between the adjustable stimulation headset 100 and the user without use of electrolytic solution, but the conductive material may be used with a electrolytic solution or gel, to provide an electrical connection between the adjustable stimulation headset 100 and the user.

Additionally, the structure of each node cluster 123 may be configured to facilitate an electrical connection between the adjustable stimulation headset 100 and the user. For example, one more or geometric parameters including a height, width, diameter, and cross-sectional profile of the plurality of contacts 126 of the node clusters 123 may be configured to facilitate the electrical connection. Each of the plurality of contacts 126 may be substantially identical to each other or may be non-identical to each other. For example, each of the plurality of contacts 126 of the node clusters 123 may be characterized by different heights, different widths, different diameters, different cross-sectional profiles, different material compositions, different mechanical behavior, different electrical behavior, any other suitable property difference, and/or any suitable combination of property differences, that facilitate an electrical connection between the adjustable stimulation headset 100 and the user. In an example, each of the plurality of contacts 126 may have a different height such that the plurality of contacts 126 form a concave contact area that is complimentary to the convex surface of the head of the user. The plurality of contacts 126 may be patterned on the node clusters 123. For example, the plurality of contacts 126 may be any one or more of a rectangular pattern, a polygonal pattern, a circular pattern, an ellipsoidal pattern, an amorphous pattern, and any other suitable pattern.

Each of the plurality of contacts 126 may have any geometric shape. Further, each of the plurality of contacts 126 may have a rotational axis of symmetry (e.g., as in a conical, screw, auger, or barb-tipped contact), a single axis of symmetry, multiple axes of symmetry (e.g., as in a pyramidal or prismatic contact), or any other suitable symmetry or asymmetry.

Each of the plurality of contacts 126 may have elastic or plastic properties that allows each of the plurality of contacts 126 to deflect and/or be deformed. Deflection or deformation of the plurality of contacts 126 may facilitate the electrical connection between the adjustable stimulation headset 100 and the user.

The plurality of contacts 126 of each node cluster 123 are intended to be non-invasive, such that the plurality of contacts 126 does not penetrate (or perforate) the user's head.

Further, each electrode of the first set of one or more pairs of electrodes 120 and second set of one or more pairs of electrodes 121 may have a dedicated ground to reduce noise. The adjustable stimulation headset 100 may be configured to select which electrodes of the first set of one or more pairs of electrodes 120 and second set of one or more pairs of electrodes 121 are ground, or become either an anode or cathode, and over a stimulation cycle the headset, the adjustable stimulation headset 100 may alternate the electrodes between anode and cathode. The ability to alternate the anode and cathode can increase the number and variety of potential stimulation patterns available for the adjustable stimulation headset 100 to apply.

In some embodiments, each electrode of the first set of one or more pairs of electrodes 120 is either an anode or a cathode. In some embodiments, at least one electrode of the first set of one or more pairs of electrodes 120 is an anode. In some embodiments, at least one electrode of the first set of one or more pairs of electrodes 120 is a cathode. In some embodiments, all electrodes of the first set of one or more pairs of electrodes 120 are either an anode or a cathode. In some embodiments, each electrode of the second set of one or more pairs of electrodes 121 is either an anode or a cathode. In some embodiments, at least one electrode of the second set of one or more pairs of electrodes 121 is an anode. In some embodiments, at least one electrode of the second set of one or more pairs of electrodes 121 is a cathode. In some embodiments, all electrodes of the second set of one or more pairs of electrodes 121 are either an anode or a cathode.

The distance between anode and cathode may be based on the current of a desired transcranial stimulation. For example, the distance between the anode and cathode may be decreased to provide a lower current that achieves a lower level of electrical stimulation. Conversely, the distance between the anode and cathode may be increased to provide a higher current that achieves a greater level of electrical stimulation. The distance between the anode and cathode may be from about 1 centimeter (cm) to about 15 cm. The distance between the anode and cathode may be 1 cm, 1.25 cm. 1.5 cm, 2 cm, 2.3 cm, 2.6 cm, 2.9 cm, 3 cm, 3.5 cm, 4 cm, 5 cm, 6 cm, 8 cm, 10 cm, 15 cm, 20 cm, or any values therebetween. Further, the distance between the anode and cathode may alter the pathways by which the current is transmitted between them, thus increasing the number and variety of potential stimulation patterns.

The adjustable stimulation headset 100 may include a mechanism for adjusting the distance between the set of one or more pairs of electrodes 120 and second set of one or more pairs of electrodes 121 and by extension the distances between the anode and cathode may be adjusted. For example, the first adjustable band 110 and the second adjustable band 111 may comprise an expandable portion thereby allowing the first set of one or more pairs of electrodes 120 and second set of one or more pairs of electrodes 121 to move laterally.

The electrodes may comprise additional mechanisms for facilitating an electrical connection between the adjustable stimulation headset 100 and the user. For example, the electrodes or node clusters 123 may pivot or swivel on a hinge to provide an adequate contact angle between the plurality of contacts 126 and user's head. The adjustability of the electrodes minimizes the distance between the plurality of contacts and the one or more regions of the user's head to be stimulated, therefore a lower charge density is required to stimulate the one or more regions of the user's head as compared with a fixed electrode system.

The adjustable stimulation headset 100 may further comprise a circuitry 150 electronically connected to the first set of one or more pairs of electrodes 120 and the second set of one or more pairs of electrodes 121. The circuitry 150 may be configured to generate a waveform for transcranial stimulation of the user. The circuitry 150 may be configured to transmit the waveform to the first set of one or more pairs of electrodes 120 and the second set of one or more pairs of electrodes 121 for transcranial stimulation of the first and second regions of the user's head. The circuitry 150 may be configured for transmission of transcranial Direct Current Stimulation (tDCS), transcranial Alternating Current Stimulation (tACS), transcranial Random Noise Stimulation (tRNS), transcranial Variable Frequency Stimulation (tVFS), transcranial Complex Waveform Stimulation (tCWS), or combinations thereof. The circuitry 150 is configured to alternate between Direct Current Stimulation (tDCS), transcranial Alternating Current Stimulation (tACS), transcranial Random Noise Stimulation (tRNS), transcranial Variable Frequency Stimulation (tVFS), and transcranial Complex Waveform Stimulation (tCWS) during a stimulation cycle. The circuitry 150 may configured to generate either a pulsed or continuous stimulation. The circuitry 150 may be configured to reverse a direction of stimulation. The ability to reverse the current direction can increase the number and variety stimulation patterns.

In some cases, the circuitry 150 generates a transcranial Direct Current Stimulation (tDCS) in the form of a small direct constant current. During transcranial Direct Current Stimulation (tDCS), the circuitry 150 is configured to generate a current having an intensity from about 0.5 milliamperes (mA) to about 2 mA. The current for transcranial Direct Current Stimulation (DCS) may have an intensity of about 0.5 mA, 0.6 mA, 0.7 mA, 0.8 mA, 0.9 mA, 1 mA, 1.2 mA, 1.4 mA, 1.6 mA, 1.8 mA, 2 mA, and any values therebetween.

The transcranial stimulation device may produce a modular simulated alternating current to deliver Transcranial Alternating Current Stimulation (tACS) and Transcranial Random Noise Stimulation (tRNS) in different sequential patterns. The modular simulated alternating current may comprise one or more waveforms such as sine, square, triangle, and sawtooth waves.

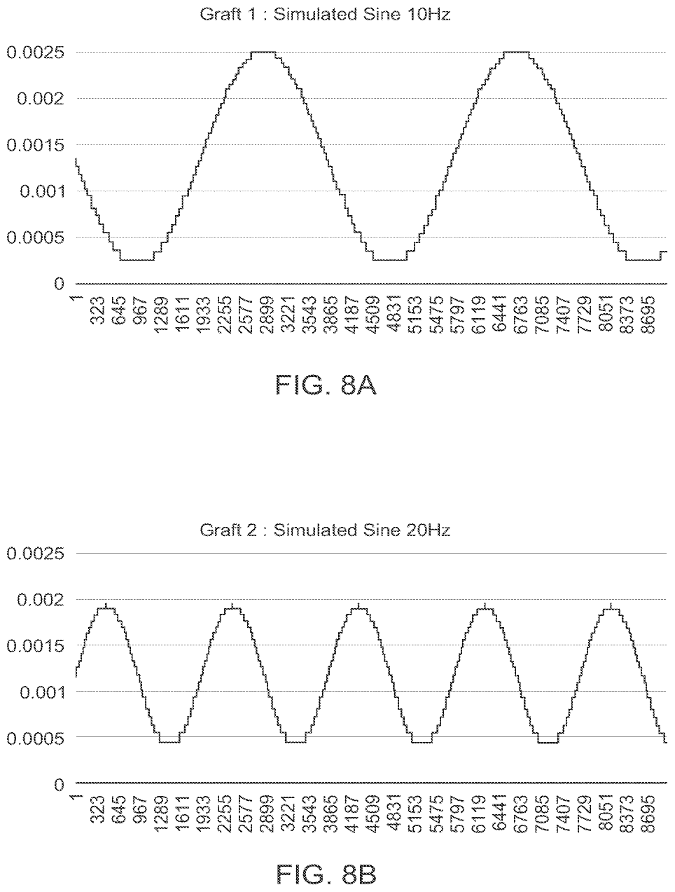

In other cases, the circuitry 150 generates a transcranial Alternating Current Stimulation (tACS) in the form of a bidirectional, biphasic current in sinusoidal waves. During transcranial Alternating Current Stimulation (tACS) the circuitry 150 is configured to generate a current with an average intensity from about 0.25 mA to about 1 mA. The current for transcranial Alternating Current Stimulation (tACS) may have an average intensity of about 0.1 mA, 0.2 mA, 0.3 mA, 0.4 mA, 0.5 mA, 0.6 mA, 0.8 mA, 1 mA, and any values therebetween. Furthermore, during transcranial Alternating Current Stimulation (tACS) the circuitry 150 is configured to generate a current having a frequency from about 1 hertz (Hz) to about 45 Hz. The current for transcranial Alternating Current Stimulation (tACS) may have a frequency of about 1 Hz, 2 Hz, 3 Hz, 5 Hz, 7 Hz, 10 Hz, 13 Hz, 17 Hz, 23 Hz, 30 Hz, 40 Hz, 45 Hz, and any values therebetween. FIGS. 8a and 8b illustrates a non-limiting example sine wave generated by the circuitry 150. FIG. 8B illustrates a non-limiting example of a modulated sine wave generated by the circuitry 150. The sine waves can be modulated to change the frequency and the amplitude.

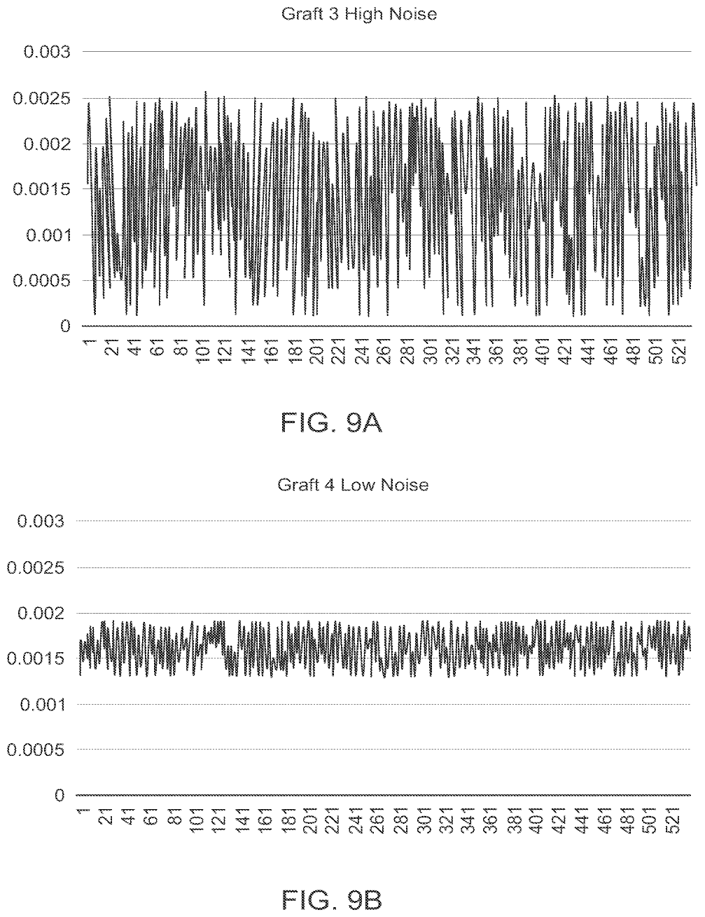

In other cases, the circuitry 150 generates a transcranial Random Noise Stimulation (tRNS) in the form of an alternating current with random amplitude and frequency. The circuitry 150 is configured to generate a current with an average intensity from about-500 microamperes (μA) to about +500 μA for transcranial Alternating Current Stimulation (tACS). FIGS. 9a and 9b illustrate a non-limiting example of random noise intensity and amplitude modulation generated by the circuitry 150.

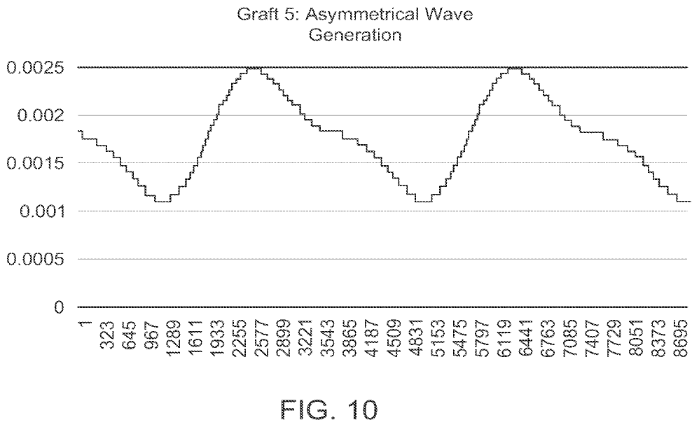

Further the circuitry 150 may be configured to generate a transcranial Complex Waveform Stimulation (tCWS) comprising any one or more of: a direct current (DC), an alternating current (AC), a random noise (RN), an AC component superimposed on a DC component, an AC component superimposed on a RN component, an RN component superimposed on a AC component, multiple AC components superimposed on each other, a monophasic pulsatile waveform, a symmetrical biphasic pulsatile waveform, an asymmetrical biphasic pulsatile waveform, and any other suitable stimulation profile. The transcranial Complex Waveform Stimulation (tCWS) may comprise continuously changing waveforms that do not have a set periodic pattern, they can be directly based on or influenced by real time biometric data received (ie brainwaves) The complex waveforms may have an average intensity from about 0.5 mA to about 5 mA, a frequency from about 0 Hz to about 150 Hz, and a minimum pulse width of 1 microsecond (μm). FIG. 10 is a non-limiting example of a complex waveform generated by the circuitry 150.

In other cases, the circuitry 150 generates a Variable Frequency Stimulation (tVFS) in the form of taking alternating currents and complex waveforms that are periodic and adjusting the period of the waveform. The waveforms can be adjusted up to 150 Hz and down to 1 Hz.

The waveform may be defined by any one of a frequency, step size, amplitude, duration, and RMS value. The circuitry 150 is configured to modulate one or more of a frequency, amplitude, duration, RMS value, and centerline offset of the waveform for transcranial stimulation of the user to produce waveforms having modulated amplitudes, modulated frequencies, and modulated pulse durations (e.g., modulated parameters characterized by exponential decay, exponential growth, or any other suitable growth or decay profiles).

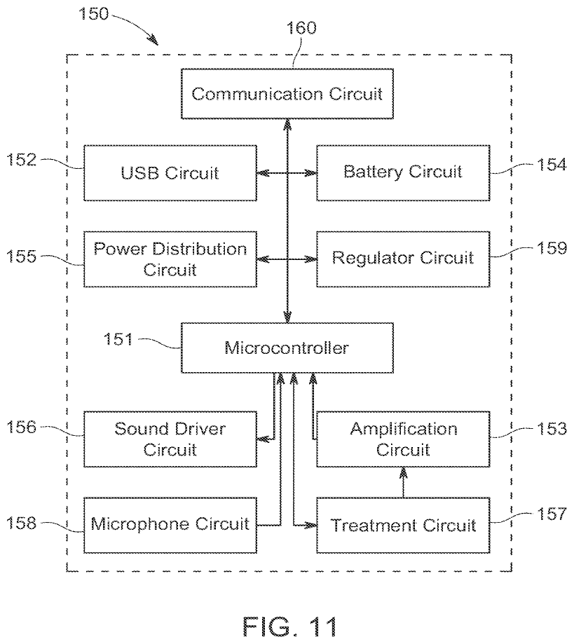

As shown in FIG. 11, the circuitry 150 may comprise a microcontroller 151, a USB circuit 152, a amplification circuit 153, a battery circuit 154, a power distribution circuit 155, a sound driver circuit 156, a treatment circuit 157, a microphone circuit 158, a regulator circuit 159, and a communication circuit 160. The components shown in the circuitry 150 may be connected to one another via a wired connection, a wireless connection (e.g., Bluetooth, Wi-Fi, near-field communication (NFC)), or a combination of both. Also, although not shown in FIG. 11, the bus may include one or more logic gates to control the data path(s). Further, although certain components are shown in the embodiment of circuitry 150, embodiments are not limited thereto, and additional components may be added and/or removed, depending on embodiments.

The device communicates with a computing device or computer (not shown) using the communication circuit 160. The communication circuit 160 may include a Bluetooth circuit. Instructions for transcranial stimulation may be provided by the user via the computer and transmitted to adjustable stimulation headset 100 using the communication circuit 160. The instructions may include activating and deactivating stimulation and/or defining one or more parameters for stimulation. Further, the computer may transmit software updates, audio data, visual data, and other forms of data to the adjustable stimulation headset 100. Further, the computer may provide instructions to the user via an app for positioning the first and second set of one or more pairs of electrodes on the first and second regions of the user's head based on a desired transcranial stimulation.

The adjustable stimulation headset 100 may be configured to collect quantitative or biometric data which is transmitted to the computer using the communication circuit 160 which in turn may upload the quantitative data to a database (not shown). Each electrode may independently be configured to detect and measure biometric data from the user. For example, the electrodes may measure biometric data such as brain waveform activity (i.e., electroencephalogram (EEG) data) at or near the stimulation region. Additional biometric data may be collected by electrical leads positioned at locations behind, near, or around the ear. The additional biometric data may include brain wave patterns resonating throughout the entire brain and physiological vitals, such as heart rate. The electrical leads may be located at a point of contact between the left headphone assembly 101 and the user, the right headphone assembly 102 and the user.

The computer may allow the user to input qualitative data which is transmitted to either or both the adjustable stimulation headset 100 and the database. The qualitative data may comprise responses by a user to a survey provided to the user through an app on the computer. The survey responses may be one or more of the user's mood, activity, quality of life, experience, attention, concentration, coordination, information processing, activity speed, or other subjective measurable data. The survey may ask the user to provide feedback on their perception of the neuromodulation (i.e., is the user experiencing an improvement in mood, activity, quality of life, etc.).

The computer may process the qualitative and/or quantitative data using a machine learning algorithm to determine a desired transcranial stimulation. The machine learning algorithm may use quantitative data (e.g., biometric data) to establish a baseline for each stimulation cycle (i.e., treatment); provide recommendations for adjusting the positioning of the first set one or more pairs of electrodes 120 and/or second set of one or more pairs of electrodes 121 to minimize a distance between the node clusters 123 and a determined treatment area (i.e., a region of the user's head); and determine a treatment schedule and duration (i.e., length of a stimulation cycle). The machine learning algorithm may provide instructions to the microcontroller 151 via the computer to adjust one or more parameters for stimulation. The adjusted parameters may include any one of a frequency, step size, amplitude, duration, and RMS value. The machine learning algorithm may provide instructions to a user via a app on the computer to adjust the position of either or both the first set of one or more pairs of electrodes 120 and second set of one or more pairs of electrodes 121.

The communication circuit 160 is electronically connected to the microcontroller 151. The communication circuit 160 may transmit instructions from the computer to the microcontroller 151 to activate and deactivate stimulation and/or define one or more parameters for stimulation. Additionally, the communication circuit 160 may transmit audio data, visual data, or other forms of data from the computer to the microcontroller 151.

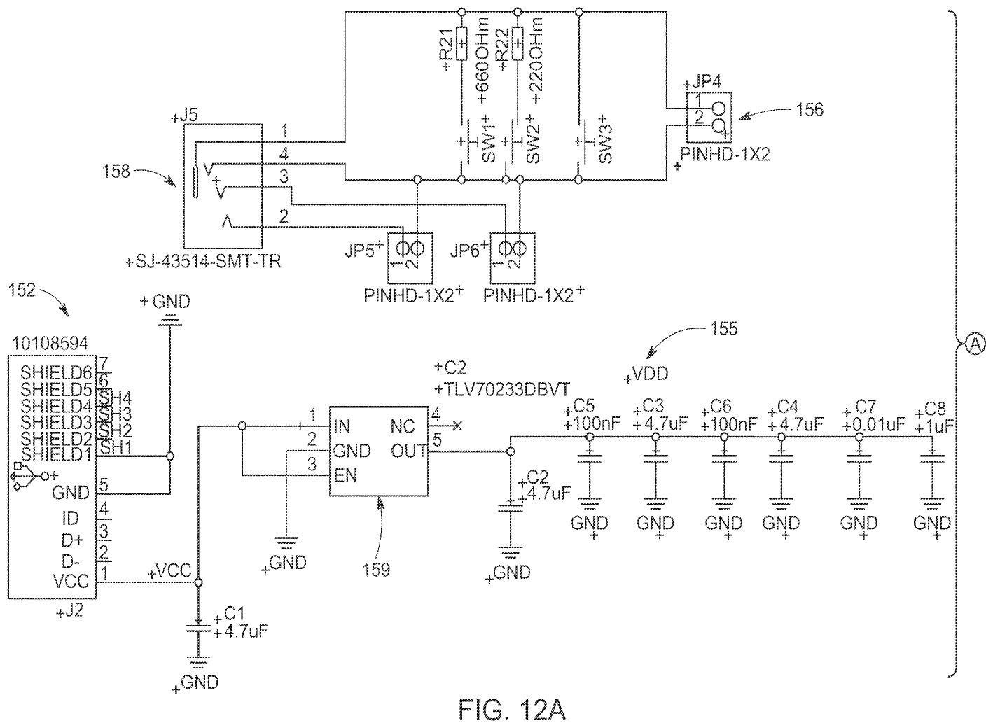

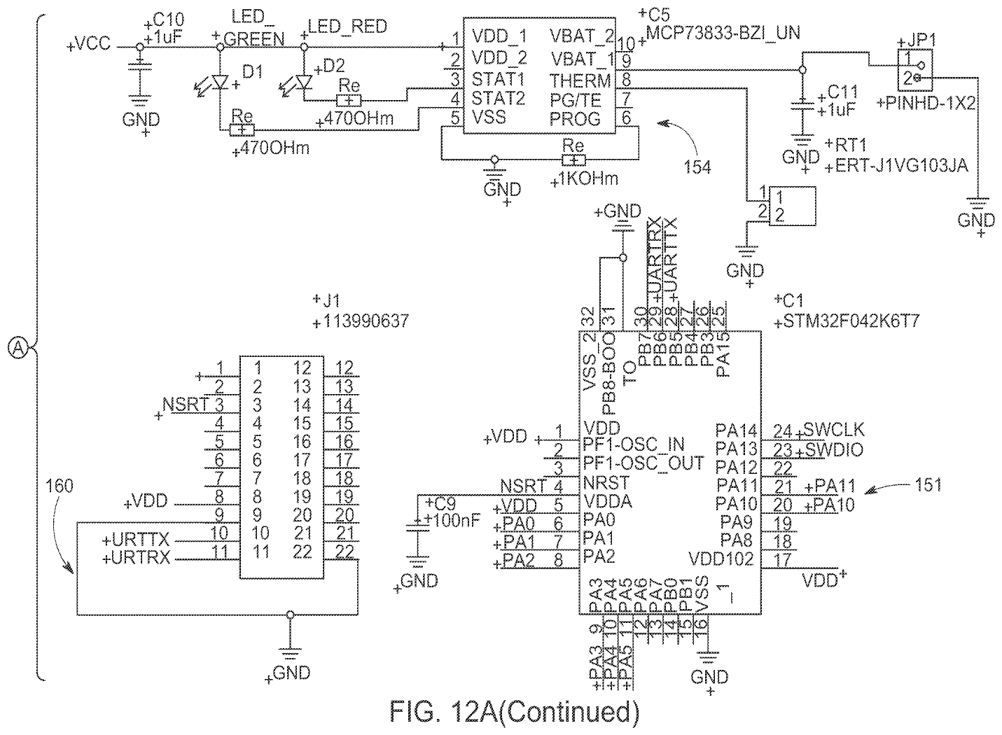

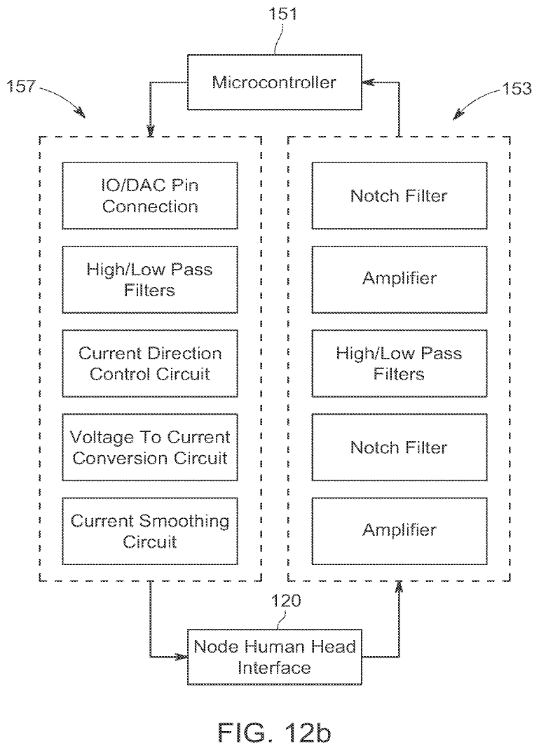

The microcontroller 151 may be electronically connected with the sound driver circuit 156 and transmits audio data from the communication circuit 160 to the sound driver circuit 156. The sound driver circuit 156 can provide audio signals to one or more speakers located in the left headphone assembly 101 and right headphone assembly 102. The microcontroller 151 may be electronically connected with the microphone circuit 158 and may receive audio data from the microphone circuit 158. The microphone circuit 158 may be coupled to one or more microphones (not shown) located on either or both the left headphone assembly 101 and right headphone assembly 102 The microcontroller 151 is electronically connected to the regulator circuit 159 which regulates the battery voltage (e.g., to about 3.3 volts (V)) via the battery circuit 154 and powers the microcontroller 151. The USB circuit 152 may interface with the battery circuit 154 to charge the battery 170 (e.g., to about 4.7 V). The USB circuit 152 may act as a secondary power source for the circuitry 150. The USB circuit 152 may be compatible with an any known USB standard (e.g., micro-USB, USB 2.0, USB 3.0, USB 3.1, etc.). For example, a USB cable may be connected to the USB circuit 152 to convey power to the different components of the circuitry 150 via the USB circuit 152. FIG. 12a shows exemplary circuitry for the regulator circuit 159, battery circuit 154, USB circuit 152, microcontroller 151, sound driver circuit 156, microphone circuit 158, communication circuit 160, and power distribution circuit 155.

The microcontroller 151 is electronically connected to and powers the treatment circuit 157 which generates the transcranial Direct Current Stimulation (tDCS), transcranial Alternating Current Stimulation (tACS), transcranial Random Noise Stimulation (tRNS), transcranial Variable Frequency Stimulation (tVFS), transcranial Complex Waveform Stimulation (tCWS), or combinations thereof. The treatment circuit 157 delivers the transcranial stimulation to the user via the first set of one or more pairs of electrodes 120 and/or second set of one or more pairs of electrodes 121. The microcontroller 151 is electronically connected with the amplification circuit 153 which may increase the electrical strength of and filter quantitative (e.g., biometric) data received by each node cluster 123. The amplification circuit 153 may also be connected to additional leads, to collect additional quantitative or biometric data (e.g., brainwaves and vitals). The additional leads may be positioned anywhere on the head of the user including behind the ear. As shown in FIG. 12b the amplification circuit 153 may consist of different types of amplification and filtering circuits such as notch filters and high or low pass filters. The voltage and the driving resistance may determine how much current is output by the treatment circuit 157. For example, if the driving resistance is low, the output current may be high, and if the driving resistance is high, the output current may be low. The voltage applied from the microcontroller 151 can change resulting in different current outputs by the treatment circuit 157. For example, a lower voltage results in a lower current, and a higher voltage results in a higher current. The driving resistance may be defined by the one or more parameters for stimulation transmitted from the computer via the communication circuit 160. The microcontroller 151 can track the current applied to or by the first set of one or more pairs of electrodes 120 and second set of one or more pairs of electrodes 121 as feedback from the treatment circuit 157. Using an internal reference, the microcontroller 150 can verify proper positioning of the first set of one or more pairs of electrodes 120 at the first region of the user's head and the second set of one or more pairs of electrodes 121 at the second region of the user's head.

When the applied current deviates from one or more threshold in the instructions provided by the computer, the microcontroller 151 may deactivate or alter (e.g., modulate) the stimulation generated by the treatment circuit 157. The microcontroller 151 may control or actuate solid state switches in the treatment circuit 157 to convert the electrodes between anode and cathode. For example, an electrode within the first set of one or more pairs of electrodes 120 may be an anode in a first configuration and a cathode in a second configuration, and a corresponding electrode may be a cathode in the first configuration and an anode in the second configuration. The microcontroller 151 may oscillate the current from positive to negative and vice versa (e.g., switch anode/cathodes as described), as well as change an amplitude of the current. The treatment circuit 157 may comprise one or more transistors (e.g., a metal-oxide-semiconductor field-effect transistor (MOSFET), a bipolar junction transistor (BJT), etc.) that regulates the current to maintain a voltage at a specific value (or within a range) allowing the device to accurately control the current. The one or more transistors may regulate the voltage(s) of the first set of one or more pairs of electrodes 120 and the second set of one or more pairs of electrodes 121. The treatment circuit 157 may open and/or close (e.g., activate or deactivate; electrically connect or disconnect) additional resistor circuits in parallel with the driving resistor. As the treatment circuit 157 closes the resistor circuits, the resistance of the driving resistor decreases in resistance allowing more current to be driven through the first set of one or more pairs of electrodes 120 and second set of one or more pairs of electrodes 121 while the one or more transistors maintains the voltage across the system. The treatment circuit 157 is electronically connected to the microcontroller 151 and is controlled by the microcontroller 151. The treatment circuit 157 executes programs to apply DC treatment (e.g., treatment with DC current), simulated AC treatment (e.g., treatment with AC current), and simulated noise treatment.

The circuitry 150 can be powered by a battery 170, which power to the adjustable stimulation headset 100. The battery 170 may include a rechargeable battery (e.g., lithium-ion, nickel-cadmium, nickel metal hydride, or lithium-ion polymer). Alternatively, the battery 170 is not a rechargeable battery. The battery 170 is configured to have any appropriate profile such that the battery 170 provides adequate power characteristics (e.g., cycle life, charging time, discharge time, etc.) for stimulation using the adjustable stimulation headset 100.

The adjustable stimulation headset 100 may be configured to collect quantitative (e.g., biometric data). Each electrode may independently be configured to detect and measure quantitative (e.g., biometric data) from the user connecting to the amplification circuit 153. For example, the electrodes may measure biometric data such as brain waveform activity (i.e., electroencephalogram (EEG) data) at or near the stimulation region. Additional biometric data may be collected by electrical leads positioned at locations behind, near, or around the ear. The additional biometric data may include brain wave patterns resonating throughout the entire brain and physiological vitals, such as heart rate. The electrical leads may be located at a point of contact between the left headphone assembly 101 and the user, the right headphone assembly 102 and the user. The quantitative data may be relayed to a computer via the communication circuit 160.

In some instances, the adjustable stimulation headset 100 may comprise audio components for use before, during, and after stimulation cycles. The audio components may include a right headphone assembly 102, and a left headphone assembly 101. The left headphone assembly 101 and the right headphone assembly 102 further include a sound driver assembly. The sound driver assembly includes an earpad, a sound driver cover, a sound driver, a sound driver housing. The left headphone assembly 101 further includes a ball joint, the circuitry 130, and a left headphone cover. The right headphone assembly 102 further includes a ball joint, the battery 170 and a right headphone cover.

The sound driver assembly of the right headphone assembly 102 and a left headphone assembly 101 may be symmetrical. The earpad may be a softcover that is configured to fit over the ear od the user and is designed for comfort while wearing the adjustable stimulation headset 100. The earpad may connect to the device using a slipcover to the sound diver cover. The sound cover driver can hold the shape of the earpad and may connect to the sound diver housing with 4 plastic wedge connectors. Within the sound driver housing may be the sound driver and the female ball joint connector 141. The sound driver may generate and provide audio signals to the user. The female ball joint connector 141 can allow for 3-dimensional rotational motion of the sound driver assembly. The 3-dimensional rotational motion may provide two benefits, namely it allows the earpad to fit flat against the user's head to optimize comfort and allows for the rotation of the first adjustable band 110 and second adjustable band 111 to allow for precise positioning of the first set of one or more pairs of electrodes 120 and second set of one or more pairs of electrodes 121. The headphone housing of the left headphone assembly 101 may contain a mall ball joint connector 140 and circuitry 150 The headphone housing of the right headphone assembly 102 may contain a male ball joint connector 140 and the battery 170. Both left and right headphone housings may interface with the left headphone cover and the right headphone cover via wedge connectors.

In some embodiments, adjustable stimulation headset 100 is modular with capability to add audio, visual, virtual reality (VR), altered reality (AR), mixed reality (MR), and other modules to extend its range of uses. The adjustable stimulation headset 100 may be configured to be multifunctional to allow the user to safely use the audio and microphone portions of the headset without having electrodes continually in contact with the human body. Adjustable stimulation headset 100 can be used for non-direct stimulation purposes, such as a Bluetooth audio and microphone device. In some embodiments, the adjustable stimulation headset 100 may be worn for a significantly longer duration than the assigned stimulation treatment window period.

### Methods for Transcranial Stimulation

Provided herein are methods for applying transcranial stimulation to a user. The methods are intended to provide a neuromodulation to a user of the transcranial stimulation system.

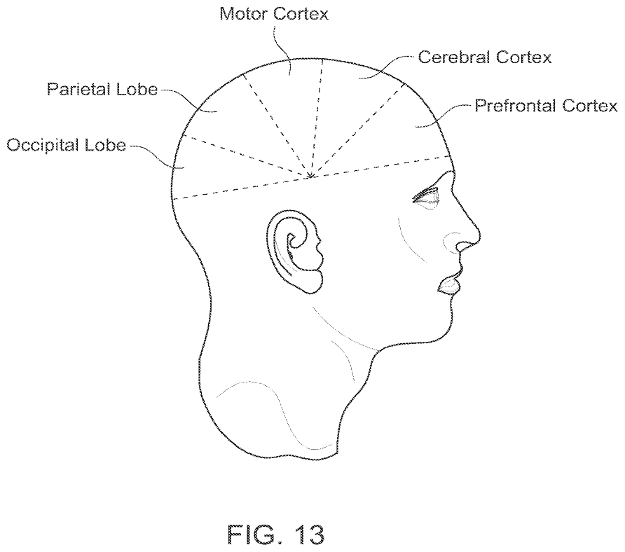

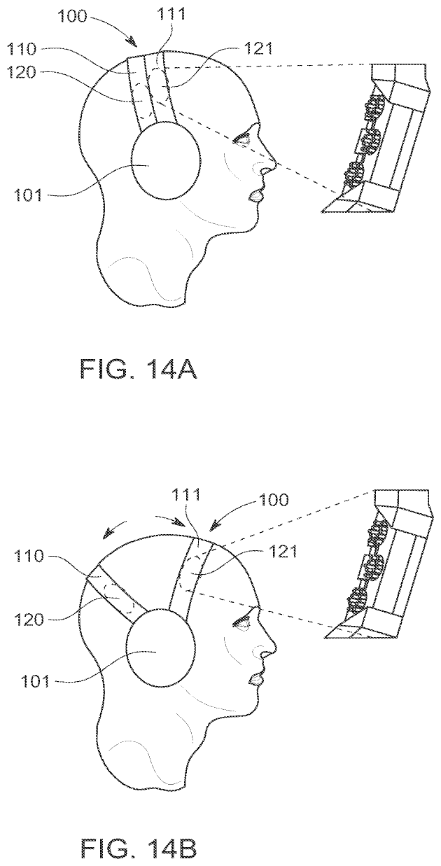

In an aspect, the method for providing transcranial stimulation to a user comprises the step of providing, to a user, instructions for positioning an adjustable stimulation headset onto the user's head to contact a first and a second region of the user's head. FIGS. 13, 14a and 14b illustrate the positioning and application of the adjustable stimulation headset 100. As shown in FIG. 13, the first and second regions of the user's head may comprise any of a frontal lobe (prefrontal cortex), the cerebral cortex, the motor cortex, the parietal lobe, and the occipital lobe. The adjustable stimulation headset 100 may be placed onto the user's head in a collapsed state as shown in FIG. 14a and may subsequently be deployed as shown in FIG. 14b, or alternatively the adjustable stimulation headset 100 may be applied onto the user's head in a deployed state. Deployment of the adjustable stimulation headset 100 may comprise separating a first adjustable band 110 and a second adjustable band 111 of the headset.

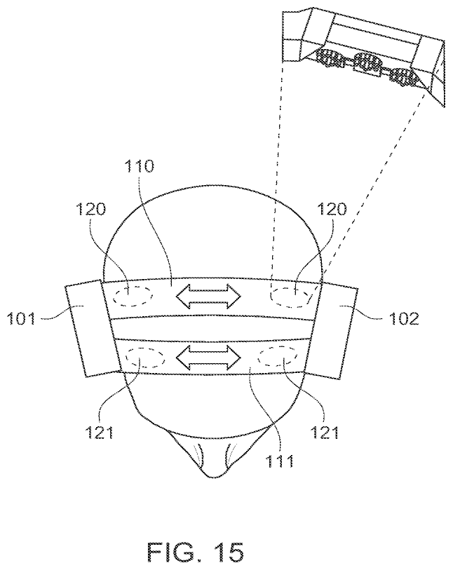

Separation of the first adjustable band 110 and second adjustable band 111 may expose a first set of one or more pairs of electrodes 120 and a second set of one or more pairs of electrodes 121. The first set of one or more pairs of electrodes 120 can be positioned onto a first region of a user's head, and the second set of one or more pairs of electrodes 121 can be positioned on a second region of a user's head. The position of the first set of one or more pairs of electrodes 120 and second set of one or more pairs of electrodes 121 may be incrementally adjusted using one or more mechanisms of the adjustable stimulation headset 100. For example, as shown in FIG. 15, the electrodes may be adjusted laterally to position the electrodes either proximal or distal to a midline of the brain.

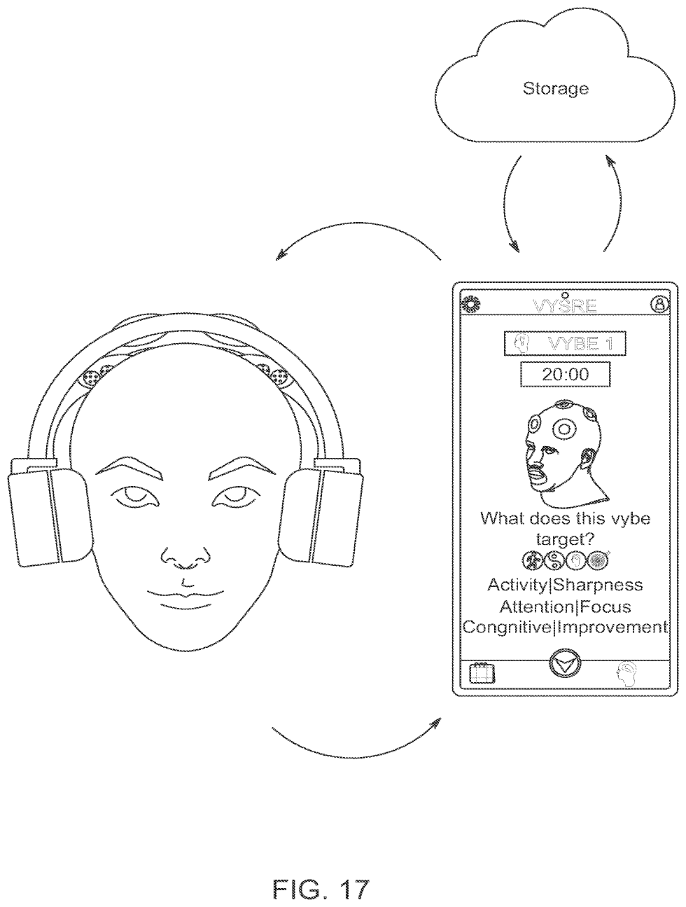

As show in FIG. 17, instructions for positioning the adjustable stimulation headset 110 may be provided to the user through an app on a computer connected to the headset. In some embodiments, the user selects a desired transcranial stimulation through the app, and the app provides instructions for positioning the adjustable stimulation headset 100 based on a desired transcranial stimulation. The instructions may indicate a position of a first set of one or more pairs of electrodes 120 of the transcranial stimulation system on a first region of the user's head, and a position of a second set of more or more pairs of electrodes 121 on a second region of the user's head. In some embodiments, the first and second regions are selected from a group comprising the frontal lobe (prefrontal cortex), the cerebral cortex, the motor cortex, the parietal lobe, and the occipital lobe.

The adjustable stimulation headset 100 may verify the position of the first set of one or more pairs of electrodes 120 and second set of one or more pairs of electrodes 121 on the first and second regions of the user's head, respectively. In verifying the position of first set of one or more pairs of electrodes 120 and second set of one or more pairs of electrodes 121, the adjustable stimulation headset 100 determines a voltage across all electrodes to ensure there is a sufficient electrical connection between the headset and the user prior to initiating a stimulation cycle. When the electrical connection between the headsets and the user is insufficient, the system may relay a signal to the user to re-position either or both of the first set of one or more pairs of electrodes 120 and second set of one or more pairs of electrodes 121. Once re-positioned, the adjustable stimulation headset 100 may reinitiate the verification to confirm whether the there is a sufficient electrical connection between the headset and the user. When there is a sufficient electrical connection between the adjustable stimulation headset 100 and the user, the headset relays a signal to the computer which prompts the user via the app to begin a stimulation cycle.

Once the transcranial stimulation system is positioned, the method for providing a transcranial stimulation to a user may comprise the step of applying the desired transcranial stimulation using the transcranial stimulation system to apply a neuromodulation to the user. The transcranial stimulation may be transcranial Direct Current Stimulation (tDCS), transcranial Alternating Current Stimulation (tACS), transcranial Random Noise Stimulation (tRNS), transcranial Variable Frequency Stimulation (tVFS), and transcranial Complex Waveform Stimulation (tCWS).

In some embodiments, transcranial Direct Current Stimulation (tDCS) is applied. The applied transcranial Direct Current Stimulation (tDCS) may an intensity from about 0.5 milliamperes (mA) to about 2 mA. The applied transcranial Direct Current Stimulation (tDCS) may have an intensity of about 0.5 mA, 0.6 mA, 0.7 mA, 0.8 mA, 0.9 mA, 1 mA, 1.2 mA, 1.4 mA, 1.6 mA, 1.8 mA, 2 mA, and any values therebetween.

In some embodiments, transcranial Alternating Current Stimulation (tACS) is applied. The applied transcranial Alternating Current Stimulation (tACS) may have an average intensity from about 0.25 mA to about 1 mA. The applied transcranial Alternating Current Stimulation (tACS) may have an average intensity of about 0.1 mA, 0.2 mA, 0.3 mA, 0.4 mA, 0.5 mA, 0.6 mA, 0.8 mA, 1 mA, and any values therebetween. Furthermore, the applied transcranial Alternating Current Stimulation (tACS) may have a frequency from about 1 hertz (Hz) to about 45 Hz. The applied transcranial Alternating Current Stimulation (tACS) may have a frequency of about 1 Hz, 2 Hz, 3 Hz, 5 Hz, 7 Hz, 10 Hz, 13 Hz, 17 Hz, 23 Hz, 30 Hz, 40 Hz, 45 Hz, and any values therebetween.

In some embodiments, transcranial Random Noise Stimulation (tRNS) is applied. The applied transcranial Random Noise Stimulation (tRNS) may have an average intensity from about −500 microamperes (uA) to about +500 μA.

In some embodiments, the applied stimulation comprises a complex waveform comprising any one or more of: a direct current (DC), an alternating current (AC), an AC component superimposed on a DC component, a monophasic pulsatile waveform, a symmetrical biphasic pulsatile waveform, an asymmetrical biphasic pulsatile waveform, and any other suitable stimulation profile. The applied complex waveform may have an average intensity from about 0.5 mA to about 5 mA, a frequency from about 0 Hz to about 150 Hz, and a minimum pulse width of 1 microsecond (μm).

The transcranial stimulation may be applied bilaterally. Bilateral stimulation comprises applying an independent wave form to each of the left and right hemispheres of the brain separately. In some embodiments, bilateral stimulation comprises applying independently to each of the left and right hemispheres either transcranial direct current stimulation (tDCS), transcranial alternating current stimulation (tACS), transcranial random noise stimulation (tRNS), transcranial variable frequency stimulation (tVFS), and transcranial Complex Waveform Stimulation (tCWS). In some embodiments, bilateral stimulation comprises applying a different transcranial stimulation to the left and right hemispheres of the user's head (i.e., brain). Additionally, transcranial stimulation may be applied cross laterally. Cross lateral stimulation comprises applying a first wave form to a first hemisphere of the user's brain which crosses or intersects with a second wave form applied to a second region of the users head.

Further, the method for providing transcranial stimulation to a user comprises the step of receiving one or more of a qualitative or quantitative feedback from the user in response to the transcranial stimulation (i.e., neuromodulation). In some embodiments, the receiving quantitative feedback may collecting biometric data such as brain waveforms (i.e., electrical activity from the brain of the user) or measuring physiological vitals of the user such as heart rate, respiration rate, temperature, etc. The biometric data may be collected prior to, during, or after the stimulation cycle. The quantitative feedback may be retrieved from the user either before, during, or after a stimulation cycle. In some embodiments, receiving quantitative feedback occurs prior to applying the desired transcranial stimulation. In some embodiments, receiving quantitative feedback occurs during transcranial stimulation. In some embodiments, receiving quantitative feedback occurs after applying the desired transcranial stimulation. The quantitative data may be transmitted from the adjustable stimulation headset 100 to the computer, which may upload the quantitative data to a database.

The qualitative feedback may include a survey provided to the user through the app on the computer. The survey may be provided to the user prior to, during, or after the stimulation cycle. The survey requests the user to provide feedback on one or more of the user's mood, activity, quality of life, experience, attention, concentration, coordination, information processing, activity speed, or other subjective measurable data. The survey may ask the user to provide feedback on their perception of the neuromodulation (i.e., whether the user interprets an improvement in mood, activity, quality of life, etc.). The qualitative feedback may be retrieved from the user either before, during, or after a stimulation cycle. In some embodiments, receiving qualitative feedback occurs prior to applying the desired transcranial stimulation. In some embodiments, receiving qualitative feedback occurs during transcranial stimulation. In some embodiments, receiving qualitative feedback occurs after applying the desired transcranial stimulation. The computer may upload the qualitative data to a database.

Additionally, the method for providing transcranial stimulation to a user comprises the step of adjusting the transcranial stimulation in response to the quantitative and qualitative feedback.

Both the qualitative and quantitative data provide a unique brain activity report of user activity and progress at any given time. The machine learning algorithm may compile a report of the quantitative and qualitative data and can provide recommendations to the user for subsequent stimulation cycles.

The quantitative and qualitative feedback are processed by the computer using a machine learning algorithm which provides a personalized and customized adjustment to provide an enhanced experience, outcome, targeted result for the user. The machine learning algorithm may use the qualitative and quantitative data to establish a baseline which may be used to measure the efficacy of each stimulation cycle. Depending the extent of user improvement over the baseline, the machine learning algorithm may utilize real-time or historical data (e.g., quantitative and qualitative feedback stored on the database) to determine one or more parameters of the transcranial stimulation to adjust, such as wave form or target stimulation region. The one or more of a qualitative or quantitative data may be weighted by the machine learning algorithm. In determining the adjustment for the one or more parameters the machine learning algorithm may weight the quantitative and qualitative data to increase the accuracy of the user recommendations. In some embodiments, the machine learning algorithm may weight the quantitative feedback greater than the qualitative back. In some embodiments, the machine learning algorithm may weight the quantitative feedback less than the qualitative feedback. In some embodiments, the machine learning algorithm may weight the quantitative feedback equally to the qualitative feedback.

The machine learning algorithm may recommend adjusting one or more parameters of the transcranial stimulation wave form. The one or more parameters may include one or more of a frequency, amplitude, duration, RMS value, and centerline offset of a transcranial stimulation wave form. Additionally, the one or more parameters may include a type (e.g., transcranial Direct Current Stimulation (tDCS), transcranial Alternating Current Stimulation (tACS), transcranial Random Noise Stimulation (tRNS), transcranial Variable Frequency Stimulation (tVFS), and transcranial Complex Waveform Stimulation (tCWS)) or direction of stimulation (e.g., bilateral, cross lateral, or reverse).

Further, the machine learning algorithm may recommend adjusting one or more parameters of the transcranial stimulation target region. The one or more parameters may include a position of either or both the first set of one or more pairs of electrodes 120 and second set of one or more pairs of electrodes 121, or a brain region to be stimulated. For example, if the user's brain wave forms and survey responses suggest a need for improved hand-eye coordination, the machine learning algorithm may determine that the motor cortex should be stimulated.

Once machine learning algorithm identifies a recommended adjustment, the algorithm can either provide instructions to the adjustable stimulation headset 100 to modulate the waveform and/or provides instructions to the user via the app on the computer to adjust the position of the first set of one or more pairs electrodes 120 and/or second set of one or more pairs of electrodes 121.

In an aspect, the method for providing transcranial stimulation to a user comprises the step of applying the adjusted desired transcranial stimulation with the adjustable transcranial stimulation device.

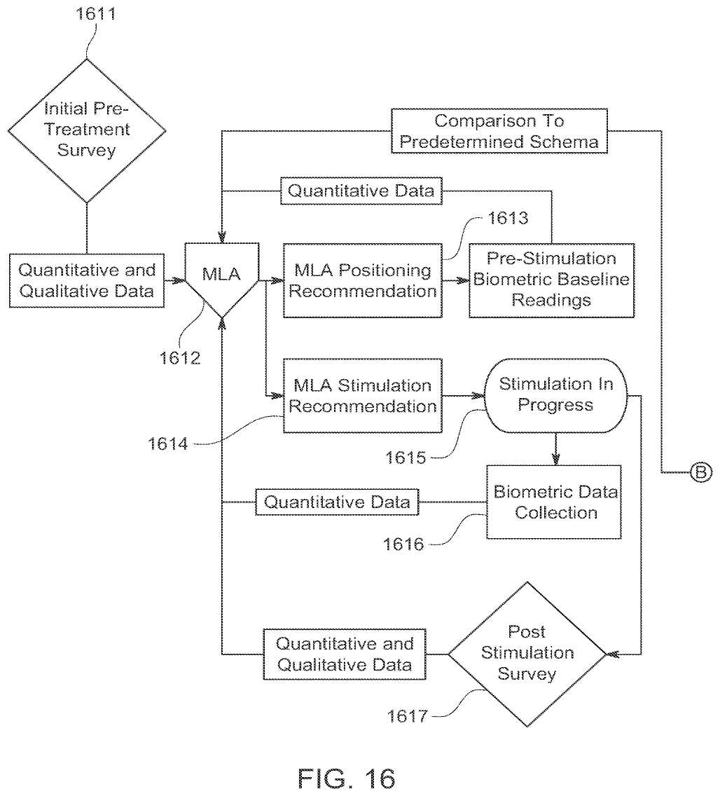

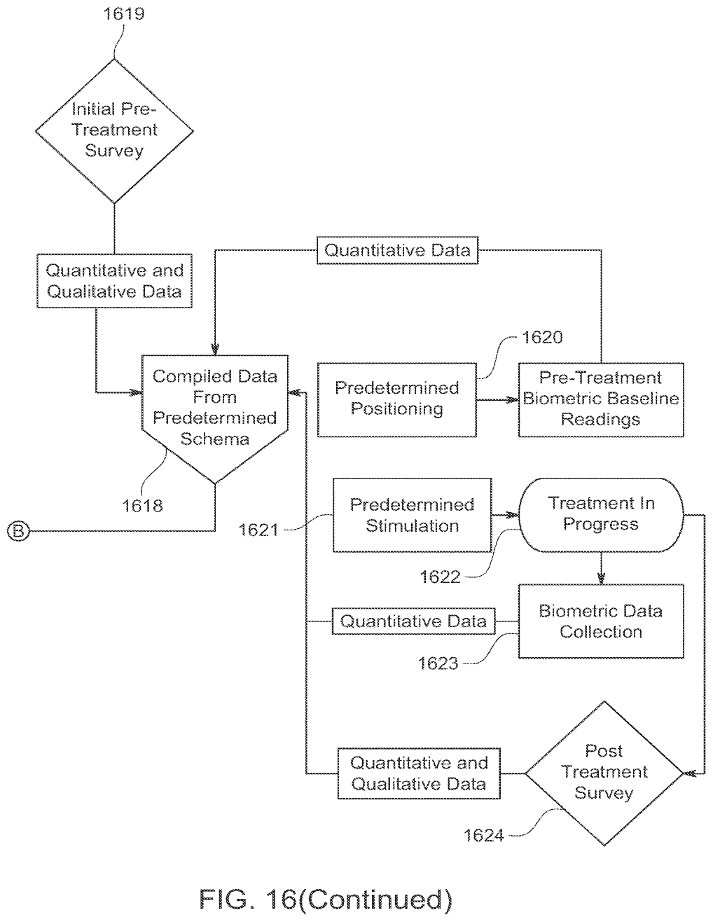

FIG. 16 is a flow chart illustrating an exemplary transcranial stimulation method 1600. At step 1611, the computer may retrieve initial pre-treatment data from the user via the adjustable stimulation headset 100. The pre-treatment data may consist of qualitative and/or quantitative data. The qualitative data may consist of a survey provided to the user via and app on the computer, and the quantitative data may consist of biometric data collected from the user using the headset. At step 1612, the computer uses a machine learning algorithm (MLA) to determine a recommended position 1613 of first set of one or more pairs of electrodes 120 and/or second set of one or more pairs of electrodes 121 based on the qualitative and/or quantitative data retrieved at step 1611. The adjustable stimulation headset 100 may retrieve additional biometric data once the electrodes are positioned which is transmitted to the computer and processed by the machine learning algorithm (MLA) to determine whether the electrodes are in a correct position based on the recommendations provided at step 1613. The machine learning algorithm (MLA) may determine a recommended stimulation 1614 including a stimulation type (i.e., tDCS, tACS, tCWS ect.), current density, and duration based on the qualitative and/or quantitative data retrieved at step 1611. At step 1615, stimulation (i.e., treatment) is initiated by the user via a command prompt provided by an app on the computer.

Once treatment (i.e., stimulation) begins, the computer may retrieve quantitative data (i.e., biometric data) 1616 from the adjustable stimulation headset 100 to monitor treatment progress and other parameters of stimulation including electrical readings provided by the circuitry 150. Stimulation may be halted in accordance with the duration recommended by the MLA at step 1614. At step 1617, the system may retrieve post-stimulation data consisting of quantitative data or qualitative data.

The machine learning algorithm (MLA) may compile the quantitative (i.e., biometric) and qualitative (i.e., survey) data 1618, including both real-time and historical data, to adjust one or more parameters of the stimulation cycle such as the stimulation location. The machine learning algorithm (MLA) may compile the quantitative and qualitative data to adjust the stimulation type (i.e., tDCS, tACS, tCWS ect.), current density, and duration in future stimulation sessions.

The recommendations provided by the machine learning algorithm (MLA) may be evaluated by comparison to a pre-determined treatment (i.e., stimulation) schema. The schema is generated by applying stimulation to the user using the adjustable stimulation headset 100 based on predetermined treatment locations 1620 and a predetermined stimulation 1621, including stimulation type (ie tDCS, tACS, tCWS ect.), current density, duration, and stimulation schedule. Prior to initiating stimulation, the computer collects quantitative data (i.e., pre-treatment biometric baseline readings) from the adjustable stimulation headset 100. At step 1622, stimulation (i.e., treatment) is initiated by the user via a command prompt provided by an app on the computer. Once treatment (i.e., stimulation) begins, the computer may retrieve quantitative data (i.e., biometric data) 1623 from the adjustable stimulation headset 100 to monitor treatment progress and other parameters of stimulation including electrical readings provided by the circuitry 150. Stimulation may be halted in accordance with the predetermined stimulation 1621. At step 1624, the system may retrieve post-stimulation data consisting of quantitative data or qualitative data (i.e., post-treatment survey). The data collected prior, during, and after stimulation is used to form the pre-determined treatment schema.

This schema may not be adjusted based off user data (i.e., data collected from the user). This schema may use or be adjusted based on a small population of user data. This schema may be used to evaluate whether the one or more parameters of the stimulation cycle adjusted by the machine learning algorithm (MLA) provided an improvement to user.

Although the above steps show method 1600 of treating a patient in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as beneficial to the treatment.

One or more of the steps of the method 1600 may be performed with the circuitry 150 as described herein. The circuitry 150 may be programmed to provide one or more of the steps of the method 1600, and the program may comprise program instructions stored on a computer readable memory.

The methods described herein are intended to cause a neuromodulation in a user. In some embodiments, the neuromodulation comprises enhancing the user's coordination or concentration. In some embodiments, the neuromodulation comprises enhancing either a brain's wellness or a brain's health. In some embodiments, the neuromodulation comprises enhancing a brain's ability to process information. In some embodiments, the neuromodulation comprises enhancing the user's task performance or acuity.

### Computing System

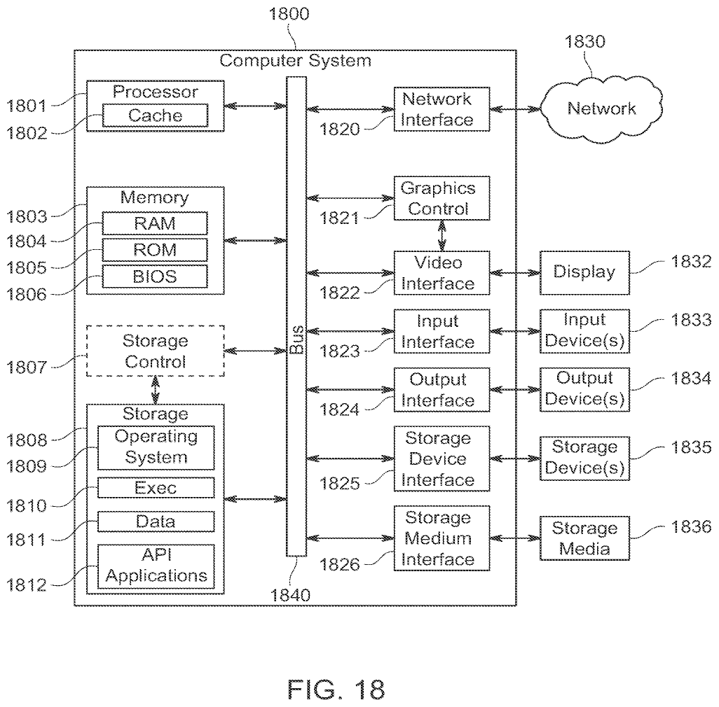

Referring to FIG. 18, a block diagram is shown depicting an exemplary machine that includes a computer system 1800 (e.g., a processing or computing system) within which a set of instructions can execute for causing a device to perform or execute any one or more of the aspects and/or methodologies for static code scheduling of the present disclosure. The components in FIG. 18 are examples only and do not limit the scope of use or functionality of any hardware, software, embedded logic component, or a combination of two or more such components implementing particular embodiments.

Computer system 1800 may include one or more processors 1801, a memory 1803, and a storage 1808 that communicate with each other, and with other components, via a bus 1840. The bus 1840 may also link a display 1832, one or more input devices 1833 (which may, for example, include a keypad, a keyboard, a mouse, a stylus, etc.), one or more output devices 1834, one or more storage devices 1835, and various tangible storage media 1836. All of these elements may interface directly or via one or more interfaces or adaptors to the bus 1840. For instance, the various tangible storage media 1836 can interface with the bus 1840 via storage medium interface 1826. Computer system 1800 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 1800 includes one or more processor(s) 1801 (e.g., central processing units (CPUs) or general purpose graphics processing units (GPGPUs)) that carry out functions. Processor(s) 1801 optionally contains a cache memory unit 1802 for temporary local storage of instructions, data, or computer addresses. Processor(s) 1801 are configured to assist in execution of computer readable instructions. Computer system 1800 may provide functionality for the components depicted in FIG. 18 as a result of the processor(s) 1801 executing non-transitory, processor-executable instructions embodied in one or more tangible computer-readable storage media, such as memory 1803, storage 1808, storage devices 1835, and/or storage medium 1836. The computer-readable media may store software that implements particular embodiments, and processor(s) 1801 may execute the software. Memory 1803 may read the software from one or more other computer-readable media (such as mass storage device(s) 1835, 1836) or from one or more other sources through a suitable interface, such as network interface 1820. The software may cause processor(s) 1801 to carry out one or more processes or one or more steps of one or more processes described or illustrated herein. Carrying out such processes or steps may include defining data structures stored in memory 1803 and modifying the data structures as directed by the software.

The memory 1803 may include various components (e.g., machine readable media) including, but not limited to, a random access memory component (e.g., RAM 1804) (e.g., static RAM (SRAM), dynamic RAM (DRAM), ferroelectric random access memory (FRAM), phase-change random access memory (PRAM), etc.), a read-only memory component (e.g., ROM 1805), and any combinations thereof. ROM 1805 may act to communicate data and instructions unidirectionally to processor(s) 1801, and RAM 1804 may act to communicate data and instructions bidirectionally with processor(s) 1801. ROM 1805 and RAM 1804 may include any suitable tangible computer-readable media described below. In one example, a basic input/output system 1806 (BIOS), including basic routines that help to transfer information between elements within computer system 1800, such as during start-up, may be stored in the memory 1803.

Fixed storage 1808 is connected bidirectionally to processor(s) 1801, optionally through storage control unit 1807. Fixed storage 1808 provides additional data storage capacity and may also include any suitable tangible computer-readable media described herein. Storage 1808 may be used to store operating system 1809, executable(s) 1810, data 1811, applications 1812 (application programs), and the like. Storage 1808 can also include an optical disk drive, a solid-state memory device (e.g., flash-based systems), or a combination of any of the above. Information in storage 1808 may, in appropriate cases, be incorporated as virtual memory in memory 1803.

In one example, storage device(s) 1835 may be removably interfaced with computer system 1900 (e.g., via an external port connector (not shown)) via a storage device interface 1825. Particularly, storage device(s) 1835 and an associated machine-readable medium may provide non-volatile and/or volatile storage of machine-readable instructions, data structures, program modules, and/or other data for the computer system 1800. In one example, software may reside, completely or partially, within a machine-readable medium on storage device(s) 1835. In another example, software may reside, completely or partially, within processor(s) 1801.

Bus 1840 connects a wide variety of subsystems. Herein, reference to a bus may encompass one or more digital signal lines serving a common function, where appropriate. Bus 1840 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 1800 may also include an input device 1833. In one example, a user of computer system 1800 may enter commands and/or other information into computer system 1800 via input device(s) 1833. Examples of an input device(s) 1833 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 1833 may be interfaced to bus 1840 via any of a variety of input interfaces 1823 (e.g., input interface 1823) including, but not limited to, serial, parallel, game port, USB, FIREWIRE, THUNDERBOLT, or any combination of the above.

In particular embodiments, when computer system 1800 is connected to network 1830, computer system 1800 may communicate with other devices, specifically mobile devices and enterprise systems, distributed computing systems, cloud storage systems, cloud computing systems, and the like, connected to network 1830. Communications to and from computer system 1800 may be sent through network interface 1820. For example, network interface 1820 may receive incoming communications (such as requests or responses from other devices) in the form of one or more packets (such as Internet Protocol (IP) packets) from network 1830, and computer system 1800 may store the incoming communications in memory 1803 for processing. Computer system 1800 may similarly store outgoing communications (such as requests or responses to other devices) in the form of one or more packets in memory 1803 and communicated to network 1830 from network interface 1820. Processor(s) 1801 may access these communication packets stored in memory 1803 for processing.

Examples of the network interface 1820 include, but are not limited to, a network interface card, a modem, and any combination thereof. Examples of a network 1830 or network segment 1830 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 1830, may employ a wired and/or a wireless mode of communication. In general, any network topology may be used.

Information and data can be displayed through a display 1832. Examples of a display 1832 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 1832 can interface to the processor(s) 1801, memory 1803, and fixed storage 1808, as well as other devices, such as input device(s) 1833, via the bus 1840. The display 1832 is linked to the bus 1840 via a video interface 1822, and transport of data between the display 1832 and the bus 1840 can be controlled via the graphics control 1821. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 1832, computer system 1800 may include one or more other peripheral output devices 1834 including, but not limited to, an audio speaker, a printer, a storage device, and any combinations thereof. Such peripheral output devices may be connected to the bus 1840 via an output interface 1824. Examples of an output interface 1824 include, but are not limited to, a serial port, a parallel connection, a USB port, a FIREWIRE port, a THUNDERBOLT port, and any combinations thereof.

In addition or as an alternative, computer system 1800 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, OracleR Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows PhoneR OS, Microsoft® Windows MobileR OS, Linux® and Palm® WebOS®. Those of skill in the art will also recognize that suitable media streaming device operating systems include, by way of non-limiting examples, Apple TV®, Roku®, BoxeeR, Google TVR, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo Wii®, Nintendo® Wii UR, and Ouya®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and OracleR. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

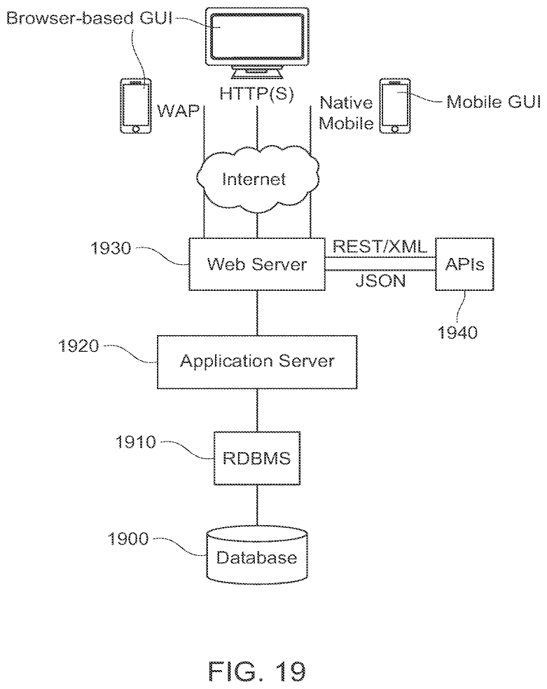

Referring to FIG. 19, in a particular embodiment, an application provision system comprises one or more databases 1900 accessed by a relational database management system (RDBMS) 2010. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 1920 (such as Java servers, .NET servers, PHP servers, and the like) and one or more web servers 1930 (such as Apache, IIS, GWS and the like). The web server(s) optionally expose one or more web services via app application programming interfaces (APIs) 1940. Via a network, such as the Internet, the system provides browser-based and/or mobile native user interfaces.

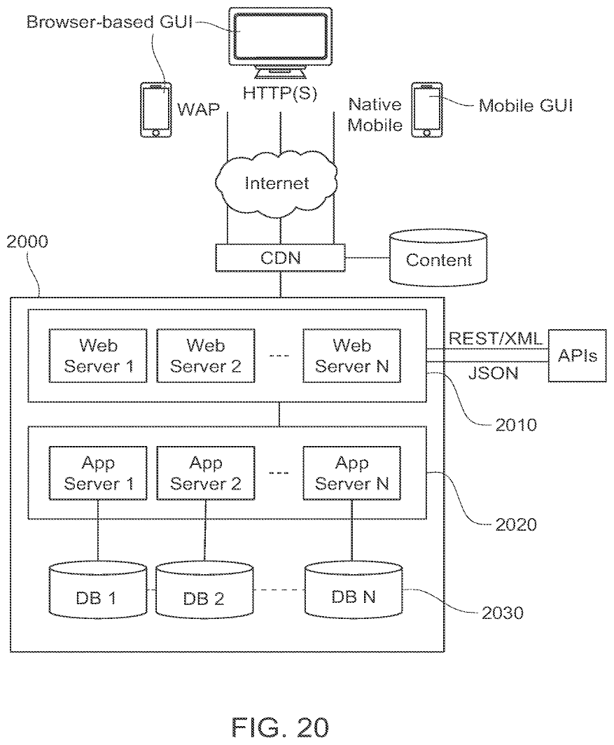

Referring to FIG. 20, in a particular embodiment, an application provision system alternatively has a distributed, cloud-based architecture 2000 and comprises elastically load balanced, auto-scaling web server resources 2010 and application server resources 2020 as well synchronously replicated databases 2030.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

### Web Browser Plug-in

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB.NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM Blackberry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of biometrics and survey (i.e., quantitative and qualitative) information. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In a particular embodiment, a database is a distributed database. In other embodiments, a database is based on one or more local computer storage devices.

### Machine Learning

In some embodiments, machine learning algorithms are utilized to aid in determining a consumer's preferred design elements. In some embodiments, the machine learning algorithm is used to detect an unpermitted renovation event, validate the detected event, or both.

In some embodiments, machine learning algorithms are utilized by the data ingestion interfaces to perform the data mining task, to detect one or more unpermitted renovation event indicia, or both. In some embodiments, machine learning algorithms are utilized by the renovation detection module to identify an initial candidate based on the detection indicia. In some embodiments, the machine learning algorithms utilized by the renovation detection module employ one or more forms of labels including but not limited to human annotated labels and semi-supervised labels. The human annotated labels can be provided by a hand-crafted heuristic. For example, the hand-crafted heuristic can comprise examining differences between public and county records. The semi-supervised labels can be determined using a clustering technique to find properties similar to those flagged by previous human annotated labels and previous semi-supervised labels. The semi-supervised labels can employ a XGBoost, a neural network, or both.

In some embodiments, machine learning algorithms are utilized by the renovation probability calculation module to calculate a probability that an unpermitted renovation event has taken or is taking place at the initial candidate. In some embodiments, the renovation probability calculation module calculates the probability that the unpermitted renovation event has taken or is taking place at the initial candidate using a distant supervision method. The distant supervision method can create a large training set seeded by a small hand-annotated training set. The distant supervision method can comprise positive-unlabeled learning with the training set as the ‘positive’ class. The distant supervision method can employ a logistic regression model, a recurrent neural network, or both. The recurrent neural network can be advantageous for Natural Language Processing (NLP) machine learning.

Examples of machine learning algorithms can include a support vector machine (SVM), a naïve Bayes classification, a random forest, a neural network, deep learning, or other supervised learning algorithm or unsupervised learning algorithm for classification and regression. The machine learning algorithms can be trained using one or more training datasets.

In some embodiments, the machine learning algorithm utilizes regression modeling, wherein relationships between predictor variables and dependent variables are determined and weighted. In one embodiment, for example, initial candidate can be a dependent variable and is derived from the detection indicia within the data set. In another embodiment, the one or more unpermitted renovation event indicia is a dependent variable derived from unique external data source. In yet another embodiment, the probability that an unpermitted renovation event has taken or is taking place at the initial candidate is a dependent variable derived from the following predictor variables: one or more unpermitted renovation event indicia, the unique external data source, and the data set.

In some embodiments, a machine learning algorithm is used to select catalogue images and recommend project scope. A non-limiting example of a multi-variate linear regression model algorithm is seen below: probability=A₀+A₁(X₁)+A₂(X₂)+A₃(X₃)+A₄(X₄)+A₅(X₅)+A₆(X₆)+A₇(X₇), wherein A₁ (A₁, A₂, A₃, A₄, A₅, A₆, A₇, . . . ) are “weights” or coefficients found during the regression modeling; and Xᵢ (X₁, X₂, X₃, X₄, X₅, X₆, X₇, . . . ) are data collected from the User. Any number of Aᵢ and Xᵢ variable can be included in the model. For example, in a non-limiting example wherein there are 7 Xᵢ terms, X₁ is the number of unpermitted renovation event indicia, X₂ is the number of initial candidates, and X₃ is the probability that an unpermitted renovation event has taken or is taking place at the initial candidate. In some embodiments, the programming language “R” is used to run the model.]

In some embodiments, training comprises multiple steps. In a first step, an initial model is constructed by assigning probability weights to predictor variables. In a second step, the initial model is used to “recommend” initial candidates. In a third step, the validation module accepts verified data regarding the unpermitted renovation event and feeds back the verified data to the renovation probability calculation. At least one of the first step, the second step, and the third step can repeat one or more times continuously or at set intervals.

In some embodiments, machine learning algorithms are utilized to aid in determining a consumer's preferred design elements. In some embodiments, the machine learning algorithm is used to determine a street address of a rental property.

In some embodiments, machine learning algorithms are utilized to determine a potential accessory dwelling unit landmark on the property. In some embodiments, machine learning algorithms are utilized to determine a potential accessory dwelling unit landmark on the property based on plurality of aerial images. In some embodiments, machine learning algorithms are utilized to determine the probability that the accessory dwelling unit can be constructed on the property. In some embodiments, machine learning algorithms are utilized to determine the probability that the accessory dwelling unit can be constructed on the property based on the potential accessory dwelling unit landmark and the plurality of property structure indicators.

In some embodiments, the machine learning algorithms herein employ one or more forms of labels including but not limited to human annotated labels and semi-supervised labels. In some embodiments, the machine learning algorithm utilizes regression modeling, wherein relationships between predictor variables and dependent variables are determined and weighted. In one embodiment, for example, the potential accessory dwelling unit landmark on the property is a dependent variable and is derived from the at least the plurality of aerial images. In another embodiment, for example, the probability that the accessory dwelling unit can be constructed on the property is a dependent variable and is derived from the potential accessory dwelling unit landmark and the plurality of property structure indicators.

The human annotated labels can be provided by a hand-crafted heuristic. For example, the hand-crafted heuristic can comprise examining differences between public and county records. The semi-supervised labels can be determined using a clustering technique to find properties similar to those flagged by previous human annotated labels and previous semi-supervised labels. The semi-supervised labels can employ a XGBoost, a neural network, or both.

In some embodiments, the potential accessory dwelling unit landmark on the property is detected using a distant supervision method. In some embodiments, the probability that the accessory dwelling unit can be constructed on the property is determined using a distant supervision method. The distant supervision method can create a large training set seeded by a small hand-annotated training set. The distant supervision method can comprise positive-unlabeled learning with the training set as the ‘positive’ class. The distant supervision method can employ a logistic regression model, a recurrent neural network, or both. The recurrent neural network can be advantageous for Natural Language Processing (NLP) machine learning.

Examples of machine learning algorithms can include a support vector machine (SVM), a naïve Bayes classification, a random forest, a neural network, deep learning, or other supervised learning algorithm or unsupervised learning algorithm for classification and regression. The machine learning algorithms can be trained using one or more training datasets.

In some embodiments, a machine learning algorithm is used to select catalogue images and recommend project scope. A non-limiting example of a multi-variate linear regression model algorithm is seen below: probability=A₀+A₁(X₁)+A₂(X₂)+A₃(X₃)+A₄(X₄)+A₅(X₅)+A₆(X₆)+A₇(X₇), wherein Aᵢ (A₁, A₂, A₃, A₄, A₅, A₆, A₇, . . . ) are “weights” or coefficients found during the regression modeling; and Xᵢ (X₁, X₂, X₃, X₄, X₅, X₆, X₇, . . . ) are data collected from the User. Any number of Aᵢ and Xᵢ variable can be included in the model. For example, in a non-limiting example wherein there are 7 Xᵢ terms, X₁ is the number of property record depictions, X₂ is the number of potential accessory dwelling unit landmarks, and X₃ is the probability that the accessory dwelling unit can be constructed on the property. In some embodiments, the programming language “R” is used to run the model.

In some embodiments, the first machine learning algorithm determines a potential accessory dwelling unit landmark on the property based on a plurality of aerial images.

In some embodiments, the first machine learning algorithm is trained by a neural network comprising: a first training module creating a first training set comprising a set of aerial images predetermined as having a potential accessory dwelling unit landmark and a set of aerial images predetermined as not having a potential accessory dwelling unit landmark; and a first training module training the neural network using the first training set; a second training module creating a second training set for second stage training comprising the first training set and the aerial images incorrectly detected as having a potential accessory dwelling unit landmark after the first stage of training; and training the neural network using the second training set.

In some embodiments, the second machine learning algorithm determines the probability that the accessory dwelling unit can be constructed on the property based on the potential accessory dwelling unit landmark and the plurality of property structure indicators. Alternatively, in some embodiments, training the first machine learning algorithm comprises multiple steps. In a first step, an initial model is constructed by assigning probability weights to predictor variables. In a second step, the initial model is used to “recommend” property structure indicators. In a third step, the validation module accepts verified data regarding the property structure indicators and feeds back the verified data. At least one of the first step, the second step, and the third step can repeat one or more times continuously or at set intervals.

In some embodiments, the second machine learning algorithm is trained by: constructing an initial model by assigning probability weights to predictor variables based on the potential accessory dwelling unit landmark and the property structure indicators; and adjusting the probability weights based on the verified data.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the invention shall also cover any such alternatives, modifications, variations or equivalents. It is intended that the following claims define the scope of the disclosure and that methods and structures within the scope of these claims and their equivalents be covered thereby.

What is claimed is:

1. A system for applying transcranial stimulation to a user, the system comprising:
a first set of one or more pairs of electrodes coupled to a first adjustable band, wherein the first adjustable band is configured to allow the first set of one or more pairs of electrodes to be positioned onto a first region of a user's head;
a second set of one or more pairs of electrodes coupled to a second adjustable band, wherein the second adjustable band is configured to allow the second set of one or more pairs of electrodes to be positioned onto a second region of the user's head, wherein the first and second regions of the user's head are selected on a desired transcranial stimulation of the user; and
a circuitry electronically connected to the first and second set of one or more pairs of electrodes, the circuitry configured to generate a waveform for the desired transcranial stimulation of the user, wherein the circuitry is configured to transmit the waveform to the first and second set of one or more pairs of electrodes to provide the desired transcranial stimulation of the first and second regions of the user's head.

2. The system of claim 1, wherein the first and second regions are selected from a group comprising the frontal lobe, prefrontal cortex, the cerebral cortex, the motor cortex, the parietal lobe, and the occipital lobe.

3. The system of claim 1, wherein the circuitry is configured to select individual electrodes of the first set of one or more pairs of electrodes and the second set of one or more pair of electrodes as inactive, active and anodic, or active and cathodic.

4-6. (canceled)

7. The system of claim 1, wherein each electrode of the first set of one or more pairs of electrodes has one or more node clusters comprising a plurality of contacts.

8. (canceled)

9. The system of claim 3, wherein each active contact of the plurality of contacts is configured to provide an electrical connection between the circuitry and the first region of the user's head.

10. (canceled)

11. The system of claim 7, wherein each electrode of the first set of one or more pairs of electrodes is adjustable to minimize a distance between the plurality of contacts and the first region of the user's head.

12. The system of claim 1, wherein each electrode of the first set of one or more pairs of electrodes is configured to measure brainwave activity near the first region of the user's head.

13. The system of claim 1, wherein the system uses an internal reference to verify proper positioning of the first set of one or more pairs of electrodes at the first region of the user's head.

14-21. (canceled)

22. The system of claim 1, wherein each electrode of the second set of one or more pairs of electrodes is configured to measure brainwave activity near the second region of the user's head.

23. (canceled)

24. The system of claim 1, wherein the system is configured to apply either transcranial direct current stimulation (tDCS), transcranial alternating current stimulation (tACS), transcranial random noise stimulation (tRNS), transcranial variable frequency stimulation (tVFS), or transcranial Complex Waveform Stimulation (tCWS).

25. The system of claim 1, wherein each electrode of the first set of one or more pairs of electrodes and each electrode of the second set of one or more pairs of electrodes is independently configured to apply either transcranial direct current stimulation (tDCS), transcranial alternating current stimulation (tACS), transcranial random noise stimulation (tRNS), transcranial variable frequency stimulation (tVFS), transcranial Complex Waveform Stimulation (tCWS).

26. The system of claim 1, wherein the system is configured to provide the desired transcranial stimulation bilaterally.

27. The system of claim 26, wherein providing the desired transcranial stimulation bilaterally comprises applying a different transcranial stimulation to the left and right hemispheres of the user's head.

28. The system of claim 27, wherein providing the desired transcranial stimulation bilaterally comprises applying, independently to each of the left and right hemispheres, either transcranial direct current stimulation (tDCS), transcranial alternating current stimulation (tACS), transcranial random noise stimulation (tRNS), transcranial variable frequency stimulation (tVFS), or transcranial Complex Waveform Stimulation (tCWS).

29. The system of claim 1, wherein the circuitry is configured to provide the desired transcranial stimulation cross laterally.

30-36. (canceled)

37. The system of claim 1, further comprising a computer configured to provide instructions to the user for positioning the first and second set of one or more pairs of electrodes on the first and second regions of the user's head.

38. The system of claim 1, wherein the position of the first and second adjustable bands relative to one another is adjustable.

39. The system of claim 1, further comprising an audio component.

40. The system of claim 1, further comprising a visual component.

41-77. (canceled)

78. The system of claim 1, further comprising a plurality of indentations configured to identify a degree of separation between the first adjustable band and the second adjustable band, wherein the degree of separation corresponds with placement of the first set of one or more pairs of electrodes and the second set of one or more pairs of electrodes relative to the first and second regions of the user's head.
